SEQUENCE LISTING (39 sequences)

SEQ ID NO: 1 - protein, 7 aa, synthetic construct
  REGION 1..7: note = CDR de nanocorps synth tique
FTSEISN

SEQ ID NO: 2 - protein, 7 aa, synthetic construct
  REGION 1..7: note = CDR de nanocorps synth tique
GTHTTQT

SEQ ID NO: 3 - protein, 12 aa, synthetic construct
  REGION 1..12: note = CDR de nanocorps synth tique
AEQDMSDLWLGS

SEQ ID NO: 4 - protein, 7 aa, synthetic construct
  REGION 1..7: note = CDR de nanocorps synth tique
YGWWIYE

SEQ ID NO: 5 - protein, 7 aa, synthetic construct
  REGION 1..7: note = CDR de nanocorps synth tique
WADGTRT

SEQ ID NO: 6 - protein, 12 aa, synthetic construct
  REGION 1..12: note = CDR de nanocorps synth tique
IYILEGTQGVPI

SEQ ID NO: 7 - protein, 7 aa, Lama glama
NIFSSNT

SEQ ID NO: 8 - protein, 8 aa, Lama glama
ISKGGSTN

SEQ ID NO: 9 - protein, 14 aa, Lama glama
NPVPDSDNYASGQG

SEQ ID NO: 10 - protein, 7 aa, Lama glama
SIFSSNV

SEQ ID NO: 11 - protein, 8 aa, Lama glama
ISRGGGTN

SEQ ID NO: 12 - protein, 13 aa, Lama glama
PIPYSGDYVSGQG

SEQ ID NO: 13 - protein, 7 aa, Lama glama
STFSMNV

SEQ ID NO: 14 - protein, 8 aa, Lama glama
IGTGGGTN

SEQ ID NO: 15 - protein, 12 aa, Lama glama
NKIPHNWGWGQG

SEQ ID NO: 16 - protein, 124 aa, synthetic construct
  REGION 1..124: note = Nanocorps synthetique
MAEVQLQASGGGFVQPGGSLRLSCAASGFTSEISNMGWFRQAPGKEREFVSAISGTHTTQTYYADSVKGRFTISRDNSKNTVYLQMNSLRAEDTATYYCAAEQDMSDLWLGSYWGQGTQVTVSS

SEQ ID NO: 17 - protein, 124 aa, synthetic construct
  REGION 1..124: note = Nanocorps synthetique
MAEVQLQASGGGFVQPGGSLRLSCAASGYGWWIYEMGWFRQAPGKEREFVSAISWADGTRTYYADSVKGRFTISRDNSKNTVYLQMNSLRAEDTATYYCAIYILEGTQGVPIYWGQGTQVTVSS

SEQ ID NO: 18 - protein, 118 aa, Lama glama
MAQVQLVESGGGLVQPGGFLRLLCTASGNIFSSNTMGWYRRAPGKQREWVASISKGGSTNYADSVKDRFTISRSITKNTVYLQMVNLKPEDTAVYYCNPVPDSDNYASGQGTQVTVSS

SEQ ID NO: 19 - protein, 118 aa, Lama glama
MAQVQLVESGGGLVPPGGSLKLSCTASGSIFSSNVMGWYRRAPGKQREWVASISRGGGTNYPDSVKGRFTVSRDIAKNTVYLQISSLKPEDTAVYYCNPIPYSGDYVSGQGTQVTVSS

SEQ ID NO: 20 - protein, 116 aa, Lama glama
MAQVQLVESGGGLVEPGGSLRLSCAVSGSTFSMNVMGWYRQAPGKHREWVGSIGTGGGTNYPDSVKGRFTISRDNVKKTVYLQMNSLKPEDTAVYYCNKIPHNWGWGQGTQVTVSS

SEQ ID NO: 21 - protein, 258 aa, synthetic construct
  REGION 1..258: note = Diabody 1 : combinaison de I5 et H4
MAEVQLQASGGGFVQPGGSLRLSCAASGFTSEISNMGWFRQAPGKEREFVSAISGTHTTQTYYADSVKGRFTISRDNSKNTVYLQMNSLRAEDTATYYCAAEQDMSDLWLGSYWGQGTQVTVSSAAAGSGSGSGSGSGSGGSQVQLVESGGGLVQPGGFLRLLCTASGNIFSSNTMGWYRRAPGKQREWVASISKGGSTNYADSVKDRFTISRSITKNTVYLQMVNLKPEDTAVYYCNPVPDSDNYASGQGTQVTVSS

SEQ ID NO: 22 - protein, 127 aa, synthetic construct
  REGION 1..127: note = Nanocorps synthetique
MAEVQLQASGGGFVQPGGSLRLSCAASGRTWSFYAMGWFRQAPGKEREFVSAISWNGGGEHYYADSVKGRFTISRDNSKNTVYLQMNSLRAEDTATYYCAHMEFSQSDYIISKWIYWGQGTQVTVSS

SEQ ID NO: 23 - protein, 124 aa, synthetic construct
  REGION 1..124: note = Nanocorps synthetique
MAEVQLQASGGGFVQPGGSLRLSCAASGSTSRDEGMGWFRQAPGKEREFVSAISRDHGWREYYADSVKGRFTISRDNSKNTVYLQMNSLRAEDTATYYCAPVIIDTMEVIPLYWGQGTQVTVSS

SEQ ID NO: 24 - protein, 121 aa, synthetic construct
  REGION 1..121: note = Nanocorps synthetique
MAEVQLQASGGGFVQPGGSLRLSCAASGATYWFSSMGWFRQAPGKEREFVSAISYWFDDIEYYADSVKGRFTISRDNSKNTVYLQMNSLRAEDTATYYCAMWNEQTGEEYWGQGTQVTVSS

SEQ ID NO: 25 - protein, 124 aa, synthetic construct
  REGION 1..124: note = Nanocorps synthetique
MAEVQLQASGGGFVQPGGSLRLSCAASGGTSFAYDMGWFRQAPGKEREFVSAISRSNSDINYYADSVKGRFTISRDNSKNTVYLQMNSLRAEDTATYYCAEAIFWGHEVIPLYWGQGTQVTVSS

SEQ ID NO: 26 - protein, 121 aa, synthetic construct
  REGION 1..121: note = Nanocorps synthetique
MAEVQLQASGGGFVQPGGSLRLSCAASGFTSARYNMGWFRQAPGKEREFVSAISRHTDRQTYYADSVKGRFTISRDNSKNTVYLQMNSLRAEDTATYYCAVGMMPKQKQYWGQGTQVTVSS

SEQ ID NO: 27 - protein, 127 aa, synthetic construct
  REGION 1..127: note = Nanocorps synthetique
MAEVQLQASGGGFVQPGGSLRLSCAASGYYFRGEDMGWFRQAPGKEREFVSAISDDSTATIYYADSVKGRFTISRDNSKNTVYLQMNSLRAEDTATYYCAPGWYIYLDWSESGSAYWGQGTQVTVSS

SEQ ID NO: 28 - protein, 121 aa, synthetic construct
  REGION 1..121: note = Nanocorps synthetique
MAEVQLQASGGGFVQPGGSLRLSCAASGYTSDLTVMGWFRQAPGKEREFVSAISSHFGPPHYYADSVKGRFTISRDNSKNTVYLQMNSLRAEDTATYYCAMRERRHSWWYWGQGTQVTVSS

SEQ ID NO: 29 - protein, 124 aa, synthetic construct
  REGION 1..124: note = Nanocorps synthetique
MAEVQLQASGGGFVQPGGSLRLSCAASGDTFNLTTMGWFRQAPGKEREFVSAISSSNTHIAYYADSVKGRFTISRDNSKNTVYLQMNSLRAEDTATYYCAYTYYGKNGKGMVYWGQGTQVTVSS

SEQ ID NO: 30 - protein, 118 aa, Lama glama
MAQVQLVESGGGLVQAGESLKLSCRMSGSTSSIQAMGWYRQTPGKQREWVASISKGGSTNYADSVKDRFTISRSITKNTVYLQMVNLKPEDTAVYYCNPVPDSDNYASGQGTQVTVSS

SEQ ID NO: 31 - protein, 118 aa, Lama glama
MAQVQLVESGGGLVQPGGSLRLSCTASGSIFSSNVMGWYRRAPGKQREWVASISRGGGTNYPDSVKGRFTVSRDIAKNTVYLQISSLKPEDTAVYYCNPIPYSGDYVSGQGTQVTVSS

SEQ ID NO: 32 - protein, 116 aa, Lama glama
MADVQLVESGGGLVQAGGSLRLSCAVSGSTFSMNVMGWYRQAPGKHREWVGSIGTGGGTNYPDSVKGRFTISRDNVKKTVYLQMNSLKPEDTAVYYCNKIPHNWGWGQGTQVTVSS

SEQ ID NO: 33 - protein, 118 aa, Lama glama
MMAQVQLVESGGGSVAAGESLTLSCRMSGSTSSIQAMGWYRRAPGNQREWLATISPRGNTNYADSVKGRFTISRDNAKNTVYLQMNSLKPADTAVYYCNTVPSGGIPWGQGTQVTVSS

SEQ ID NO: 34 - protein, 117 aa, Lama glama
MADVQLVESGGGSVPPGGSLRLSCAASGSIFTINSMGWFRQAPGNQREWLATISPRGNTNYADSVKGRFTISRDNAKNTVYLQMNSLKPADTAVYYCNTVPSGGIPWGQGTQVTVSS

SEQ ID NO: 35 - protein, 123 aa, Lama glama
MAQVQLVESGGGWVQPGGSLRLSCAASGITFSTKTMGWYRQRPGKRREWIGSISPTGFTNYADAVKGRVTISRDNGENTVYLQMNSLKPEDTAVYFCNGVPPVIIDGQSVAYWGQGTQVTVSS

SEQ ID NO: 36 - protein, 129 aa, Lama glama
MAQVQLVESGGGLVQPGGSLTLSCVISGTIFNDGSSVNAMNWYREAPGKGRVLIASLDSAGITKYADSVNGRFTISADNARETVTLQMNNLAPEDTAVYYCAATKVWNSWYATYGIDYWGKGTQVTVSS

SEQ ID NO: 37 - protein, 120 aa, Lama glama
MAQVQLVESGGGTVQAGGSLRLSCTASGRGRNAMGWYRQVPGGKRELVASINRGGTTDYTDSVKDRFTISRDSAKNTVYLQMDSLQPEDTAVYYCNAESTVIPGHIYDYWGRGTQVTVSS

SEQ ID NO: 38 - protein, 16 aa, synthetic construct
  REGION 1..16: note = peptide de liaison
GSGSGSGSGSGSGSGS

SEQ ID NO: 39 - protein, 5 aa, synthetic construct
LPETG

(19) United States
(12) Patent Application Publication
(10) Pub. No.: US 2025/0206813 A1
(43) Pub. Date: Jun. 26, 2025

(54) LIGANDS SPECIFIC FOR THE PCPE-1 GLYCOPROTEIN AND USES THEREOF

(71) Applicants: NVH MEDICINAL, Dijon (FR); David VANDROUX, Dijon (FR); Centre National de la Recherche Scientifique, Paris (FR); Université Claude Bernard Lyon 1, VILLEURBANNE (FR); CENTRE GEORGES-FRANCOIS LECLERC, Dijon Cedex (FR)

(72) Inventors: Sandrine VADON-LE GOFF, Saint-Genis-Laval (FR); Catherine MOALI, Saint-Genis-Laval (FR); Priscillia LAGOUTTE, Charnoz-sur-Ain (FR); David VANDROUX, Dijon (FR); Alexandra OUDOT, Dijon (FR)

(21) Appl. No.: 18/848,106

(22) PCT Filed: Mar. 16, 2023
(86) PCT No.: PCT/FR2023/050369

§ 371 Date: Sep. 17, 2024

(30) Foreign Application Priority Data
Mar. 17, 2022 (FR) FR2202379

(51) Int. Cl. C07K 16/18; G01N 33/574

(52) U.S. Cl. CPC C07K 16/18; G01N 33/574; C07K 2317/22; C07K 2317/35; C07K 2317/565; C07K 2317/569; C07K 2317/76; C07K 2317/92

(57) ABSTRACT

The present application relates to a ligand specific for the PCPE-1 glycoprotein, characterized in that it is a nanobody. The application also relates to a ligand specific to the PCPE-1 glycoprotein, characterized in that it inhibits its activity with the C-terminal domain of procollagens. The nanobody can include three complementarity determining regions (CDRs) with PCPE-1.

### CROSS-REFERENCE TO RELATED APPLICATIONS

The present application is a filing under 35 U.S.C. 371 as the National Stage of International Application No. PCT/FR2023/050369, filed Mar. 16, 2023, entitled “LIGANDS SPECIFIC FOR THE PCPE-1 GLYCOPROTEIN AND USES THEREOF,” which claims priority to French Application No. 2202379 filed with the Intellectual Property Office of France on Mar. 17, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4692-20000 BNT242001USPC Sequence Listing.xml; created on Sep. 11, 2024; and having a file size of 52.9 KB.

The information in the Sequence Listing is incorporated herein in its entirety for all purposes.

### FIELD OF THE INVENTION

The present invention relates to ligands specific to the PCPE-1 (Procollagen C-proteinase enhancer-1) protein. It also relates to the use thereof in medical imaging and in diagnostic and treatment methods. More specifically, it focuses on two types of ligands: some capable of binding to PCPE-1 for use in imaging, and other so-called antagonist ligands capable of binding to PCPE-1 and inhibiting its activity, for use in therapeutics. In one embodiment of the invention, these antagonist ligands are used for the diagnosis and treatment of fibrosis and cancer.

### BACKGROUND OF THE INVENTION

Fibrillar collagens are the most abundant proteins in the human body and the main components of extracellular matrices. They shape organs and tissues and play a crucial role in maintaining their homeostasis or repairing them after injury. Long considered to have a structural role in tissues, collagens are now recognized as integral players in numerous cellular processes such as cell adhesion, proliferation, migration and differentiation.

However, these collagens are also associated with numerous pathological processes and are prime targets for the development of new diagnostic tools or treatments.

Thus, fibrosis, which is characterized by excessive deposition of extracellular matrix composed mainly of collagen fibers, is a major common denominator of many pathologies that strongly affects disease progression, and efficacy and delivery of therapies (Henderson et al. 2020). Moreover, the production of abnormal collagen by tumor cells or the microenvironment plays a major role in the immune escape of these cells, and contributes strongly to their metastatic capacity as well as to their dormancy and resistance to treatment (Shi et al. 2022).

Targeting collagen and, more specifically, all the key stages involved in its biosynthesis, secretion, maturation or macro-structuring are all targets of interest for limiting the excessive collagen production associated with these processes. These include LARP6, the P4H complex, HSP47, SHMT2, LOX, etc. (Shi et al. 2022). Among the targets mentioned, those involved in the proteolytic maturation of N- and C-terminal regions seem particularly interesting. Thus, BTPs (bone morphogenetic protein-1 (BMP1)/tolloid-like proteinases) are the main proteases responsible for the removal of C-propeptides from fibrillar procollagens, a process generally considered to be the rate-limiting step in the formation of type I collagen fibrils. However, although BTPs are prime targets, in addition to collagens, they have activity directed against numerous other substrates involved in several pathways (activation of growth factors, angiogenesis, mineralization, tumor progression, etc.), which means that inhibiting their activity could lead to undesirable side effects.

Another key regulator of collagen fibrillogenesis is PCPE-1 (Procollagen C-Proteinase Enhancer-1), a secreted glycoprotein that specifically stimulates C-terminal cleavage of fibrillar procollagen by BTPs. PCPE-1 is composed of two CUB (Complement-Uegf-BMP-1) domains and a C-terminal NTR (Netrin-like) domain, separated by a long linker. The CUB domains are necessary and sufficient for PCPE-1 activity (Kronenberg 2009; Vadon 2011), which is due to their direct and close interaction with the C-propeptide of the procollagens. This interaction is thought to allow local destructuring of the procollagen trimer, which facilitates cleavage by BTPs, and explains why PCPE-1 has no effect on other BMP-1 substrates. In addition, data have described the overexpression of PCPE-1 in different fibrotic contexts (Lagoutte et al. Matrix Biol Plus 2021), making it a promising target for monitoring and/or treating fibrosis. In particular, a correlation between PCPE-1 expression levels and fibrosis, particularly cardiac fibrosis, has been established (Lagoutte et al., BioRxiv, 2021). Direct detection of PCPE-1 in biological fluids by immunoassay has also been proposed as a diagnostic method (US2012270246 for bone formation, WO2017065206A1 for NASH).

However, there is currently no robust method for non-invasively detecting PCPE-1 in tissues, and thus monitoring fibrotic collagen accumulation. Furthermore, the absence of effective tools to inhibit the action of PCPE-1 has hitherto prevented the evaluation of a pharmacological strategy targeting this protein.

International application WO 2019/080284 teaches that the PCPE-1 protein is a therapeutic target for combating fibrosis-type diseases, including skin fibrosis and difficult scarring. The strategy considered in this paper is the inhibition of PCPE-1 expression, via the injection of siRNAs designed to block translation of the gene into protein.

Nevertheless, in certain therapeutic cases, it is preferable to inhibit the activity of a protein rather than to limit its expression. To date, no drug has been reported to block the interaction between the PCPE-1 protein and procollagen C-propeptides.

For the diagnosis of cardiac fibrosis, histological analysis of cardiac biopsies remains the traditional technique, despite its invasive nature and the fact that it can only analyze a small portion of cardiac tissue, not necessarily representative of the whole. The use of MRI combined with gadolinium injection now enables imaging of large fibrotic areas, but is not completely specific for fibrosis, and does not detect interstitial fibrosis.

One of the characteristics of fibrosing pathologies and cancers is their progressive nature, which means that even if, in the case of fibrosis in particular, it is the result of stress or injury, the excessive production of collagen is continuous and only leads to pathological and functional effects at the end of a sometimes very long period. There is therefore a major need to be able to detect this excessive collagen production at an early stage and monitor its development over time. Molecular imaging, coupled with increasingly sensitive detection systems (isotopic imaging, fluorescence, etc.), is now an indispensable tool for longitudinal monitoring of these pathologies, as well as for monitoring the therapeutic effect of new treatments.

To date, the main approach adopted for direct molecular imaging of collagen, particularly in fibrotic contexts, is based on the use of probes targeting collagen directly, mainly by hybridization. Examples include collagelin peptides, EP-3533 or CBP8 (Désogère et al., 2019).

Although the results are promising, other indirect routes, such as the one disclosed in the present invention, would offer definite advantages by possibly limiting the background signal-to-noise ratio. This is particularly true of probes targeting collagen integrin receptors.

The use of immunoglobulins to target proteins of interest has been widely proven, both for therapeutic and molecular imaging purposes, and they now account for the majority of biomedicines on the market. In recent years, immunoglobulin scaffolds, devoid of the undesirable properties of conventional immunoglobulins (high molecular weight, heterotetrameric composition, disulfide bonds), have emerged as alternatives to conventional antibodies. Among them, nanobodies, also known as VHH (Variable Heavy-chain domains of Heavy-chain antibodies), derived from the heavy chain of camelid immunoglobulins, discovered in the early '90s, have demonstrated their interest in both therapeutics and diagnostics (Muyldermans, 2021). Nanobodies are the smallest antibody fragments (around 15 kDa), highly soluble and stable, and can be easily produced in large quantities in prokaryotic systems. Additionally, their convex paratope is well suited to binding to hard-to-target cavities on the antigen surface.

Three types of approach can be used to select antigen-specific nanobodies: from immune libraries, naive libraries or synthetic libraries. An immune library is generated by immunizing a camelid with the target. These are the most widely used nanobody libraries, although some targets are not immunogenic or are too toxic for animal immunization. Because of these limitations, several synthetic libraries have been developed. A selection can be made from these libraries to identify nanobodies specific to one or more target proteins. To date, over 1,400 VHHs have been successfully selected and are used as research tools, in biotechnological or diagnostic applications.

### BRIEF DESCRIPTION OF THE INVENTION

The present invention relates to a ligand specific for the PCPE-1 glycoprotein, characterized in that it is a nanobody, also designated by the abbreviation VHH (Variable domain of the Heavy chain of Heavy chain-only antibodies).

The present invention also relates to a ligand specific for the PCPE-1 glycoprotein, characterized in that it inhibits its activity. This specific ligand is also known as PCPE-1 antagonist ligand. In particular, it is a PCPE-1 antagonist nanobody.

Another object of the invention is a ligand specific for the PCPE-1 glycoprotein consisting of a combination of two or three nanobodies as described above.

The present invention also relates to a nucleic acid encoding a nanobody or a combination of two or three nanobodies, as described in the present application.

The present invention also relates to a specific antagonist ligand for the PCPE-1 glycoprotein for use as a drug, and more specifically for medical use in the treatment of cancer or fibrosis, particularly cardiac fibrosis.

The present invention also relates to a ligand specific for the glycoprotein PCPE-1 as described in the present application, characterized in that it is coupled to a detectable marker, in particular a marker used in medical imaging.

Another object of the invention is the use of such a specific ligand for the PCPE-1 glycoprotein coupled to a detectable marker, for in vivo monitoring of a pathological condition by medical imaging.

Another object of the invention is a specific ligand for the PCPE-1 glycoprotein as described above, for its theranostic use in the treatment and in vivo monitoring of a pathological condition by medical imaging.

Finally, the present application relates to a kit for determining the activity and/or amount of the PCPE-1 glycoprotein in a biological sample in vitro or ex vivo, comprising:
a ligand specific for the PCPE-1 glycoprotein as described above, and
detection reagents.

### LEGENDS OF THE FIGURES

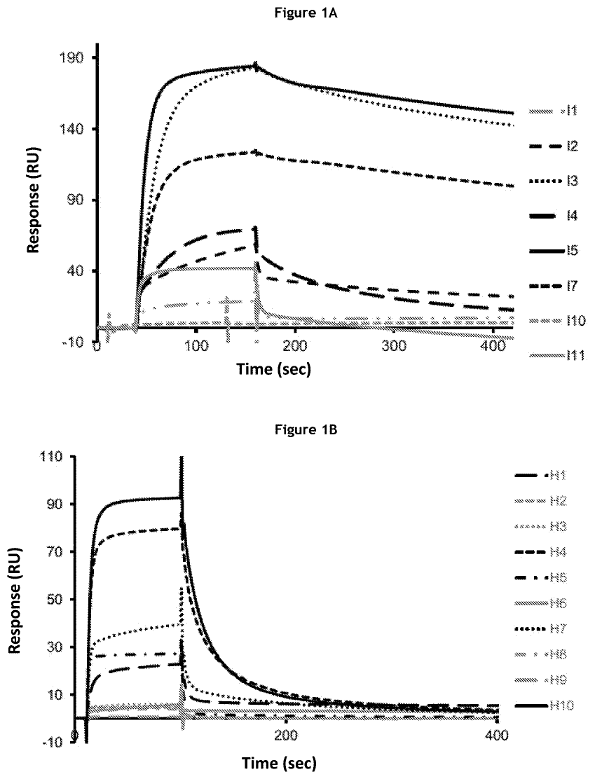

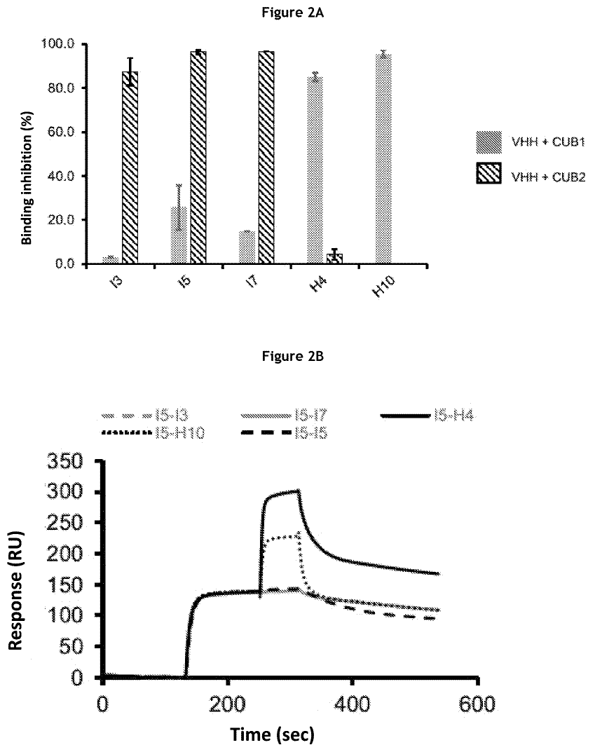

### FIG. 1: SPR Characterization of PCPE-1/VHHs Interaction

FIG. 1A: Injection of 200 nM of VHHs selected by immunization on immobilized PCPE-1 (645 RU, that is, Resonance Units).

FIG. 1B: Injection of 200 nM of VHHs selected from a synthetic library onto immobilized PCPE-1 (645 RU).

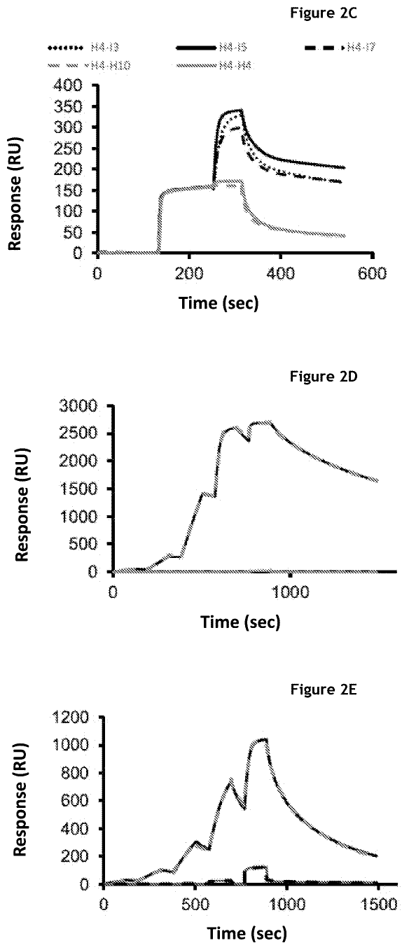

### FIG. 2: Specificity of VHHs

FIG. 2A: % inhibition of interaction between VHHs (50 nM) and immobilized PCPE-1 when the CUB1 or CUB2 domains (250 nM) are co-injected.

FIG. 2B: Injection of 250 nM VHH-I5 followed by injection of the same concentration of VHH-I5 in combination with 250 nM of a second VHH.

FIG. 2C: Injection of 250 nM VHH-H4 followed by injection of the same concentration of VHH-H4 in combination with 250 nM of a second VHH.

FIG. 2D: VHHs 15 is biotinylated and immobilized on a streptavidin chip (813 RU), then increasing concentrations of PCPE-1 (black) or PCPE-2 (grey) are injected.

FIG. 2E: VHH H4 is biotinylated and immobilized on a streptavidin chip (491 RU), then increasing concentrations of PCPE-1 or PCPE-2 are injected.
D, E: Interactions are modeled (dotted line) using the Langmuir model (Biacore T200 Evaluation software, Cytiva)

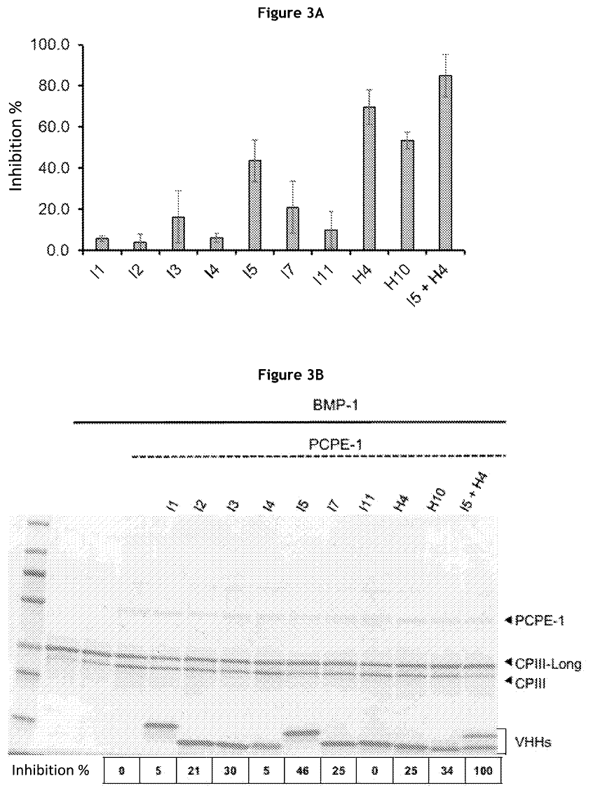

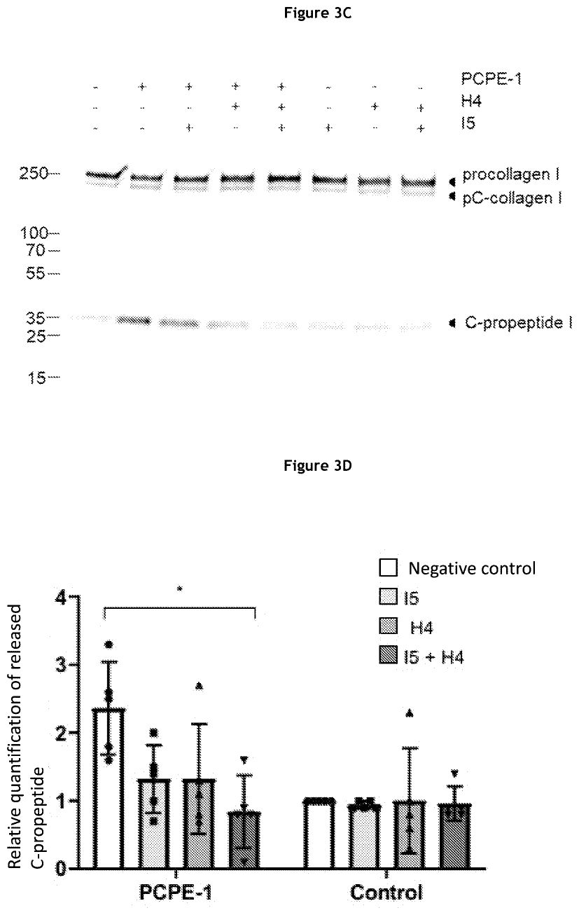

### FIG. 3: Inhibition of PCPE-1 Activity by VHHs

FIG. 3A: % inhibition of PCPE-1/mini-procollagen III interaction in the presence of VHHs: the interaction of PCPE-1 (5 nM) with immobilized mini-procollagen III is measured and compared with the interaction obtained when PCPE-1 (5 nM) is co-injected with 250 nM VHHs. Mean±SD, n=3.

FIG. 3B: Effect of VHHs on PCPE-1 activity: CPIII-Long is incubated with BMP-1 and 75 nM PCPE-1 in the absence or presence of 2 μM VHHs (1 h, 37° C.). Analysis by SDS-PAGE (4-20% gel, in the presence of DTT, Coomassie blue staining). The % inhibition is calculated by comparison with the PCPE-1 condition alone (representative gel from n=3 experiments).

FIG. 3C: Immunodetection of procollagen I and its C-terminal cleavage products in rat heart fibroblast culture medium, after 48 h of culture in the absence or presence of PCPE-1 (5 μg/mL) and VHHs (15 μg/mL). Western blot analysis using an anti-C-propeptide antibody to procollagen I (LF41).

FIG. 3D: Relative quantification of released C-propeptide, under the conditions of FIG. 3C, by densitometry (mean±SD, n=3).

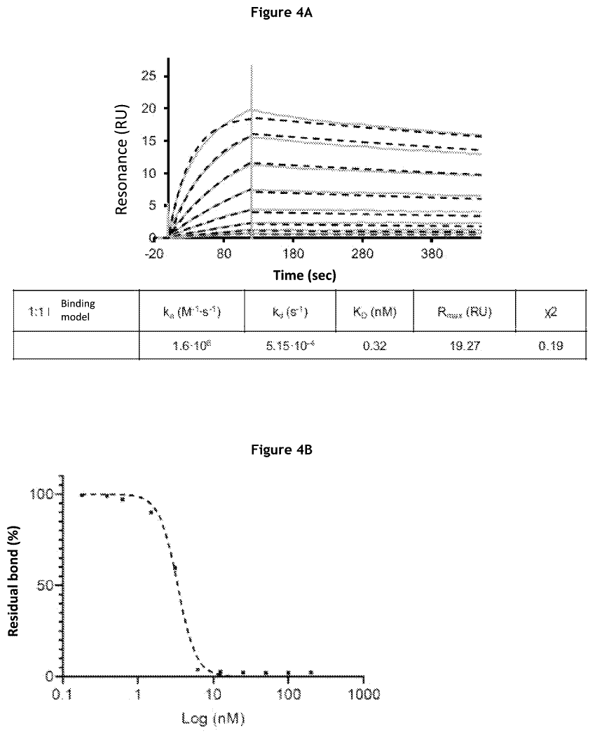

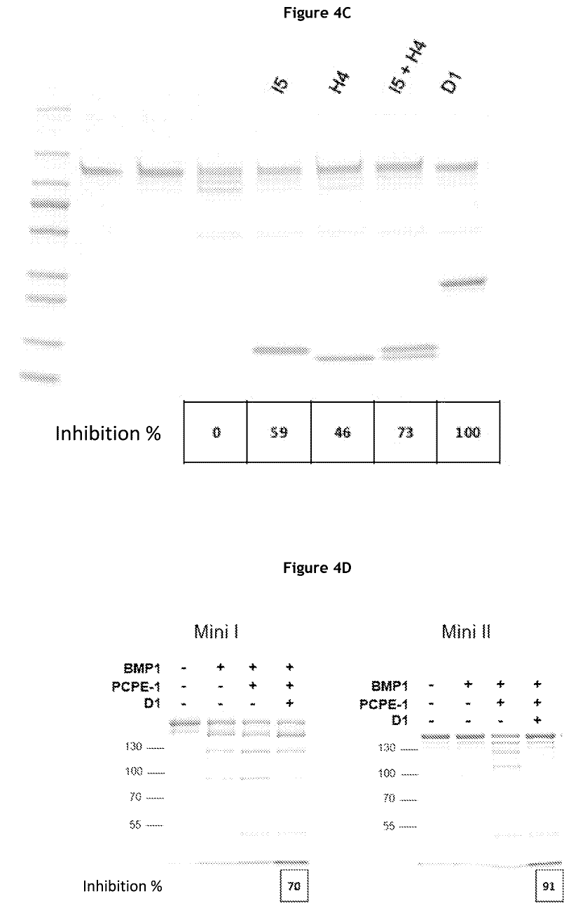

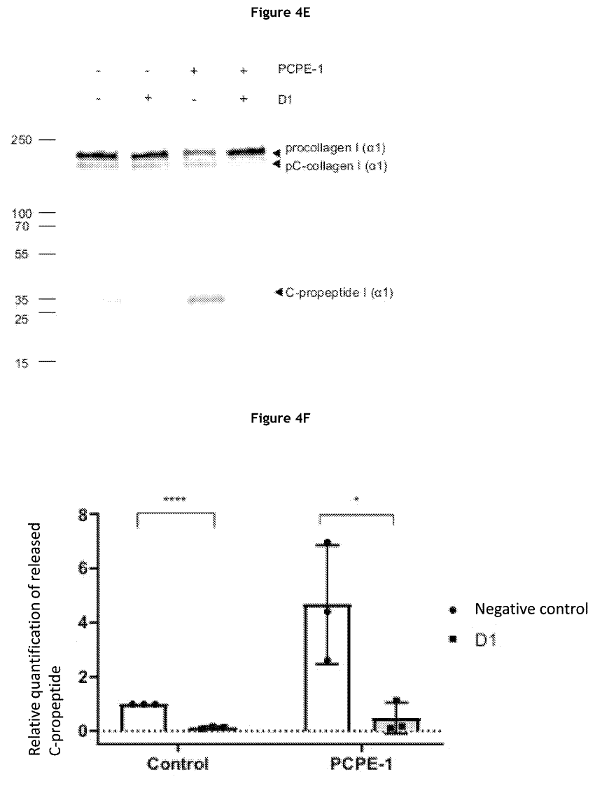

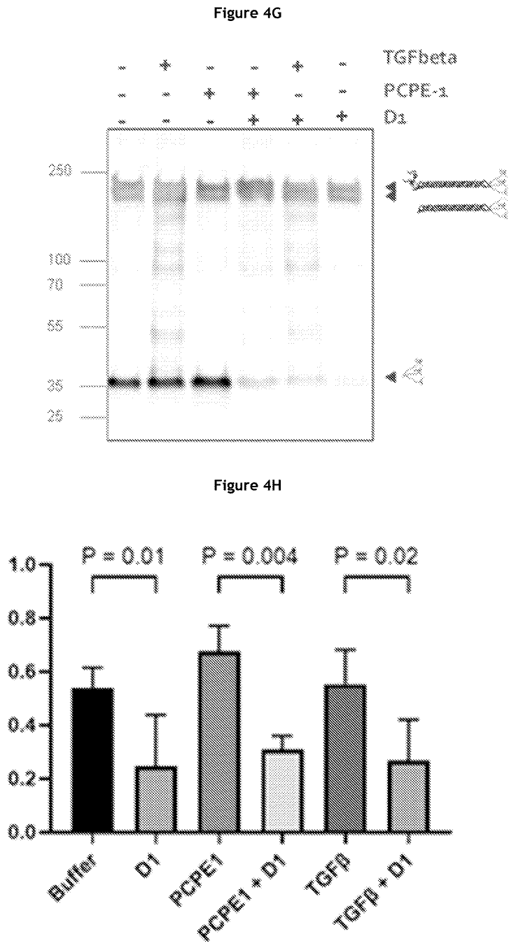

FIG. 4: Characterization of the D1 Antibody (H4-GS₈-I5) with Sequence SEQ ID NO. 21

FIG. 4A: SPR analysis of D1 interaction with immobilized PCPE-1. Sequential injections of increasing concentrations of D1 (0-250 nM) and modeling of the interaction (dotted line) using the Langmuir model (Biacore T200 Evaluation software, Cytiva).

FIG. 4B: Inhibition of PCPE-1/mini-procollagen III interaction in the presence of D1: the interaction of PCPE-1 (5 nM) with immobilized mini-procollagen III is measured and compared with the interaction obtained when PCPE-1 (5 nM) is co-injected with increasing amounts (0-200 nM) of D1. The % residual PCPE-1 uptake is plotted as a function of D1 concentration.

FIG. 4C: D1 inhibits PCPE-1 activity on procollagen III: CPIII-Long is incubated with BMP-1 and 75 nM PCPE-1 in the absence or presence of 2 μM monovalent or bivalent nanobody (1 h, 37° C.). Analysis by SDS-PAGE (4-20% gel, Coomassie blue staining). The % inhibition is calculated by comparison with the PCPE-1 condition alone.

FIG. 4D: D1 inhibits PCPE-1 activity on procollagens I and II: Mini I or Mini II are incubated with BMP-1 and 75 nM PCPE-1, in the absence or presence of 2 μM D1 (1 h, 37° C.). Analysis is performed by SDS-PAGE (8% gel, Coomassie blue staining). The % inhibition is calculated by comparison with the PCPE-1 condition alone.

FIG. 4E: Immunodetection of procollagen I and its C-terminal cleavage products in rat heart fibroblast culture medium, after 48 h of culture in the absence or presence of PCPE-1 (5 μg/mL) and the diabody D1 (15 μg/mL). Western blot analysis using an anti-C-propeptide antibody to procollagen I (LF41).

FIG. 4F: Relative quantification of released C-propeptide, under the conditions of FIG. 4E, by densitometry (mean±SD, n=3).

FIG. 4G: Immunodetection of procollagen I and its C-terminal cleavage products in human skin fibroblast culture medium, after 72 h of culture in the absence or presence of PCPE-1 (5 μg/mL), TGF-beta (5 ng/mL) and the diabody D1 (15 μg/mL). Western blot analysis using an anti-C-propeptide antibody to procollagen I (LF41).

FIG. 4H: Relative quantification of released C-propeptide, under the conditions of FIG. 4G, by densitometry (mean±SD, n=3).

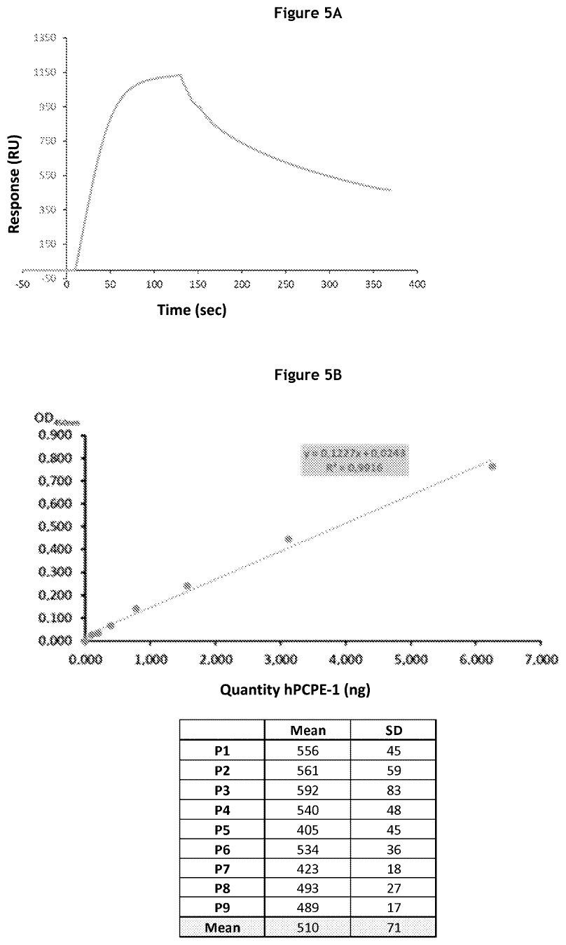

### FIG. 5: Use of a Nanobody Coupled to a Detectable Marker for PCPE-1 Detection

FIG. 5A: SPR characterization of biotinylated PCPE-1/VHH-H4 interaction. Biotinylation of VHH-H4 enables oriented capture on a streptavidin chip. The presence of PCPE-1 in the injected sample is detected by its interaction with VHH-H4.

FIG. 5B: ELISA detection: Calibration curve obtained by adding increasing quantities of purified human PCPE-1. PCPE-1 concentration (ng/ml) in human plasma samples, mean±SD of 3 determinations, each in duplicate.

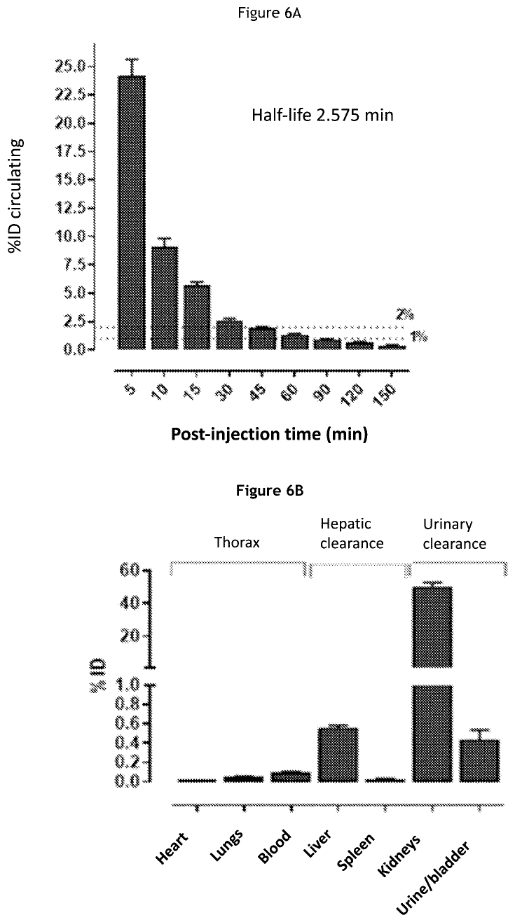

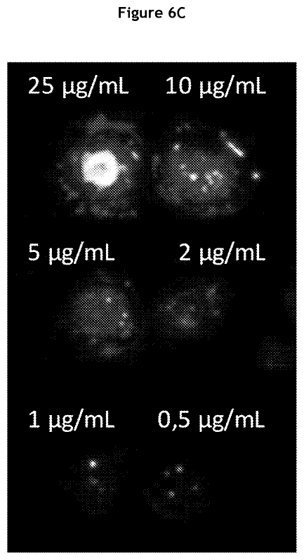

### FIG. 6: Characteristics of the VHH-H4-Ga⁶⁸ Nanobody for Imaging Applications

FIG. 6A: Pharmacokinetics of VHH-H4-Ga⁶⁸. Measurement of half-life in blood; assay during the 2.5 h post-injection, expressed as % of the injected dose (% ID, mean±SD, gamma count)

FIG. 6B: Biodistribution of VHH-H4-Ga⁶⁸ determined ex-vivo 2.5 h after injection, expressed as % of injected dose (% ID, mean±SD) for each organ (gamma count)

FIG. 6C: Detection range of VHH-H4-Ga⁶⁸ on rat heart sections. Autoradiography (phosphorimager) after one hour incubation and one hour exposure.

### DETAILED DESCRIPTION OF THE INVENTION

The present invention relates to a ligand specific for the PCPE-1 glycoprotein, characterized in that it is a nanobody (also known as VHH).

The PCPE-1 (Procollagen C-proteinase enhancer-1) glycoprotein is a key regulator of collagen fibrillogenesis. The human protein is listed in the UniProt database under reference Q15113, where its polypeptide sequence is described.

Collagens are synthesized in the form of soluble precursors known as pro-collagens. These undergo proteolytic maturation before being able to assemble into collagen fibers. This limiting step is regulated by the PCPE-1 glycoprotein, which specifically stimulates C-terminal cleavage of fibrillar procollagen by BTP proteases, responsible for the removal of C-propeptides from fibrillar procollagens. To achieve this, PCPE-1 interacts closely with the collagen C-propeptide via its two CUB (Complement-Uegf-BMP-1) domains.

The term “specific ligand” refers to a compound that interacts with a protein in a non-covalent, reversible and specific manner.

A ligand is said to be “specific” when, on the one hand, it binds preferentially to its target protein among a multitude of target proteins with similar structures; and, on the other hand, it presents an affinity, that is, a strength of interaction between said ligand and the target protein, deemed to be sufficiently strong. Affinity is quantitatively measured by the association/dissociation equilibrium constant, also known as the affinity constant or equilibrium dissociation constant, or “K_{D}” for short. The lower the K_{D} value, the higher the binding affinity between the ligand and its target protein.

Within the meaning of the present invention, a ligand is considered to bind specifically to PCPE-1 if its affinity constant K_{D} is less than 100 nM, or less than 80 nM, or less than 50 nM, and more preferentially if it is less than 30 nM. With respect to these figures, the person skilled in the art will be able to determine which ligands are specific to the invention.

A nanobody is a single-domain antibody, corresponding to an antibody fragment composed of a single monomeric variable antibody domain, which corresponds to the variable domain of the single heavy chain of antibodies of the type found in camelids, which are naturally devoid of light chains. A nanobody is able to bind selectively to a specific antigen. It has the advantage of a molecular weight of only 12 to 15 kDa, around 10 times less than common antibodies, which have a molecular weight of 150 to 160 kDa. Nanobodies also take up much less space than conventional antibodies. Additionally, this simplified structure makes the synthesis process easier, especially in bacterial cells. The first single-domain antibodies were developed from heavy-chain antibodies found in camelids; these are known as VHH fragments.

In the present application, the terms nanobody and VHH are used interchangeably and both refer to a single-domain antibody.

The nanobodies each have three CDRs, designated CDR1, CDR2 and CDR3, respectively.

The nanobodies according to the invention can in particular be llama nanobodies or synthetic nanobodies.

A combination of two covalently coupled nanobodies, with or without a binding agent, is referred to as a “diabody” or “bivalent nanobody.”

A combination of three nanobodies is known as a “tribody” or “trivalent nanobody”. Other ligands specific to the PCPE-1 glycoprotein are also included in the invention, in particular any nanobody-equivalent protein structure, excluding antibodies. Examples of such protein structures (“protein scaffold”) capable of binding specifically to target epitopes are presented in the review by (Gebauer & Skerra, 2019)

The invention also relates to a ligand specific for the PCPE-1 glycoprotein, characterized in that it inhibits its interaction with the C-terminal domain of procollagens, thereby inhibiting the activity of this PCPE-1 glycoprotein.

In addition to their ability to bind to this glycoprotein, certain PCPE-1-specific ligands have the capacity to inhibit the activity of said PCPE-1 glycoprotein. They are then referred to as “PCPE-1 antagonist ligands.”

The term “antagonist” refers to a compound that interacts with a physiologically active target protein, reducing or even suppressing the physiological activity of said protein. As shown in example 3, significant inhibition of PCPE-1 activity corresponds to:
either inhibition of its interaction with the C-terminal domains of procollagen;
or an inhibitory action on procollagen cleavage, which under normal conditions is stimulated by the action of PCPE-1.

More specifically, this inhibition of activity is characterized by inhibition of the interaction of PCPE-1 with the C-terminal domains of procollagen.

This inhibition of PCPE-1 activity is considered significant when it is greater than 40%, or even at least 50%, and preferentially greater than 60%.

These PCPE-1 antagonist ligands can be of any type, including synthetic chemical compounds, nucleic acids or protein structures, in particular polypeptide chains.

According to a preferred implementation, the PCPE-1 antagonist ligand is a polypeptide chain, and in particular is a nanobody (VHH). It is understood that any polypeptide structure equivalent to a nanobody is included in the invention.

### Specific Nanobodies

The inventors have identified two families of nanobodies with either PCPE-1 binding activity for use in medical imaging and diagnostics, or PCPE-1 binding and inhibitory activity for use as drugs.

These nanobodies were selected from a synthetic nanobody library or after llama immunization and possess PCPE-1 binding activity measured by surface plasmon resonance as shown in FIG. 1. They were selected to have a K_{D} of less than 100 nM, and more specifically less than 30 nM (see Table 2 for examples).

These nanobodies were sequenced, along with their CDRs; the sequences are presented in Table 1 below.

TABLE 1
Sequences

| SEQ ID NO. | Polypeptide sequence | Object |
|---|---|---|
| 1 | FTSEISN | CDR1 from H4 |
| 2 | GTHTTQT | CDR2 from H4 |
| 3 | AEQDMSDLWLGS | CDR3 from H4 |
| 4 | YGWWIYE | CDR1 from H10 |
| 5 | WADGTRT | CDR2 from H10 |
| 6 | IYILEGTQGVPI | CDR3 from H10 |
| 7 | NIFSSNT | CDR1 from I5 |
| 8 | ISKGGSTN | CDR2 from I5 |
| 9 | NPVPDSDNYASGQG | CDR3 from I5 |
| 10 | SIFSSNV | CDR1 from I7 |
| 11 | ISRGGGTN | CDR2 from I7 |
| 12 | PIPYSGDYVSGQG | CDR3 from I7 |
| 13 | STFSMNV | CDR1 from I3 |
| 14 | IGTGGGTN | CDR2 from I3 |
| 15 | NKIPHNWGWGQG | CDR3 from I3 |
| 16 | MAEVQLQASGGGFVQPGGSLRLSCAASGFTSEISNMGW | H4 |
| | FRQAPGKEREFVSAISGTHTTQTYYADSVKGRFTISRDNS | |
| | KNTVYLQMNSLRAEDTATYYCAAEQDMSDLWLGSYWG | |
| | QGTQVTVSS | |
| 17 | MAEVQLQASGGGFVQPGGSLRLSCAASGYGWWIYEMG | H10 |
| | WFRQAPGKEREFVSAISWADGTRTYYADSVKGRFTISRD | |
| | NSKNTVYLQMNSLRAEDTATYYCAIYILEGTQGVPIYWG | |
| | QGTQVTVSS | |
| 18 | MAQVQLVESGGGLVQPGGFLRLLCTASGNIFSSNTMGW | I5 |
| | YRRAPGKQREWVASISKGGSTNYADSVKDRFTISRSITK | |
| | NTVYLQMVNLKPEDTAVYYCNPVPDSDNYASGQGTQVT | |
| | VSS | |
| 19 | MAQVQLVESGGGLVPPGGSLKLSCTASGSIFSSNVMGW | I7 |
| | YRRAPGKQREWVASISRGGGTNYPDSVKGRFTVSRDIA | |
| | KNTVYLQISSLKPEDTAVYYCNPIPYSGDYVSGQGTQVT | |
| | VSS | |
| 20 | MAQVQLVESGGGLVEPGGSLRLSCAVSGSTFSMNVMG | I3 |
| | WYRQAPGKHREWVGSIGTGGGTNYPDSVKGRFTISRDN | |
| | VKKTVYLQMNSLKPEDTAVYYCNKIPHNWGWGQGTQV | |
| | TVSS | |
| 21 | MAEVQLQASGGGFVQPGGSLRLSCAASGFTSEISNMGW | D1 = H4 + 15 |
| | FRQAPGKEREFVSAISGTHTTQTYYADSVKGRFTISRDNS | |
| | KNTVYLQMNSLRAEDTATYYCAAEQDMSDLWLGSYWG | |
| | QGTQVTVSSAAAGSGSGSGSGSGSGGSQVQLVESGGGL | |
| | VQPGGFLRLLCTASGNIFSSNTMGWYRRAPGKQREWVA | |
| | SISKGGSTNYADSVKDRFTISRSITKNTVYLQMVNLKPED | |
| | TAVYYCNPVPDSDNYASGQGTQVTVSS | |
| 22 | MAEVQLQASGGGFVQPGGSLRLSCAASGRTWSFYAMG | H1 |
| | WFRQAPGKEREFVSAISWNGGGEHYYADSVKGRFTISR | |
| | DNSKNTVYLQMNSLRAEDTATYYCAHMEFSQSDYIISK | |
| | WIYWGQGTQVTVSS | |
| 23 | MAEVQLQASGGGFVQPGGSLRLSCAASGSTSRDEGMG | H2 |
| | WFRQAPGKEREFVSAISRDHGWREYYADSVKGRFTISR | |
| | DNSKNTVYLQMNSLRAEDTATYYCAPVIIDTMEVIPLY | |
| | WGQGTQVTVSS | |
| 24 | MAEVQLQASGGGFVQPGGSLRLSCAASGATYWFSSMG | H3 |
| | WFRQAPGKEREFVSAISYWFDDIEYYADSVKGRFTISRD | |
| | NSKNTVYLQMNSLRAEDTATYYCAMWNEQTGEEYWG | |
| | QGTQVTVSS | |
| 25 | MAEVQLQASGGGFVQPGGSLRLSCAASGGTSFAYDMG | H5 |
| | WFRQAPGKEREFVSAISRSNSDINYYADSVKGRFTISRDN | |
| | SKNTVYLQMNSLRAEDTATYYCAEAIFWGHEVIPLYWG | |
| | QGTQVTVSS | |
| 26 | MAEVQLQASGGGFVQPGGSLRLSCAASGFTSARYNMG | H6 |
| | WFRQAPGKEREFVSAISRHTDRQTYYADSVKGRFTISRD | |
| | NSKNTVYLQMNSLRAEDTATYYCAVGMMPKQKQYWG | |
| | QGTQVTVSS | |
| 27 | MAEVQLQASGGGFVQPGGSLRLSCAASGYYFRGEDMG | H7 |
| | WFRQAPGKEREFVSAISDDSTATIYYADSVKGRFTISRDN | |
| | SKNTVYLQMNSLRAEDTATYYCAPGWYIYLDWSESGSA | |
| | YWGQGTQVTVSS | |
| 28 | MAEVQLQASGGGFVQPGGSLRLSCAASGYTSDLTVMGW | H8 |
| | FRQAPGKEREFVSAISSHFGPPHYYADSVKGRFTISRDN | |
| | SKNTVYLQMNSLRAEDTATYYCAMRERRHSWWYWGQ | |
| | GTQVTVSS | |
| 29 | MAEVQLQASGGGFVQPGGSLRLSCAASGDTFNLTTMG | H9 |
| | WFRQAPGKEREFVSAISSSNTHIAYYADSVKGRFTISRDN | |
| | SKNTVYLQMNSLRAEDTATYYCAYTYYGKNGKGMVYW | |
| | GQGTQVTVSS | |
| 30 | MAQVQLVESGGGLVQAGESLKLSCRMSGSTSSIQAMGW | I1 |
| | YRQTPGKQREWVASISKGGSTNYADSVKDRFTISRSITK | |
| | NTVYLQMVNLKPEDTAVYYCNPVPDSDNYASGQGTQVT | |
| | VSS | |
| 31 | MAQVQLVESGGGLVQPGGSLRLSCTASGSIFSSNVMGW | I6 |
| | YRRAPGKQREWVASISRGGGTNYPDSVKGRFTVSRDIA | |
| | KNTVYLQISSLKPEDTAVYYCNPIPYSGDYVSGQGTQVT | |
| | VSS | |
| 32 | MADVQLVESGGGLVQAGGSLRLSCAVSGSTFSMNVMG | I8 |
| | WYRQAPGKHREWVGSIGTGGGTNYPDSVKGRFTISRDN | |
| | VKKTVYLQMNSLKPEDTAVYYCNKIPHNWGWGQGTQV | |
| | TVSS | |
| 33 | MMAQVQLVESGGGSVAAGESLTLSCRMSGSTSSIQAMG | I4 |
| | WYRRAPGNQREWLATISPRGNTNYADSVKGRFTISRDN | |
| | AKNTVYLQMNSLKPADTAVYYCNTVPSGGIPWGQGTQ | |
| | VTVSS | |
| 34 | MADVQLVESGGGSVPPGGSLRLSCAASGSIFTINSMGWF | I9 |
| | RQAPGNQREWLATISPRGNTNYADSVKGRFTISRDNAK | |
| | NTVYLQMNSLKPADTAVYYCNTVPSGGIPWGQGTQVT | |
| | VSS | |
| 35 | MAQVQLVESGGGWVQPGGSLRLSCAASGITFSTKTMG | I2 |
| | WYRQRPGKRREWIGSISPTGFTNYADAVKGRVTISRDN | |
| | GENTVYLQMNSLKPEDTAVYFCNGVPPVIIDGQSVAYWG | |
| | QGTQVTVSS | |
| 36 | MAQVQLVESGGGLVQPGGSLTLSCVISGTIFNDGSSVNA | I10 |
| | MNWYREAPGKGRVLIASLDSAGITKYADSVNGRFTISAD | |
| | NARETVTLQMNNLAPEDTAVYYCAATKVWNSWYATYGI | |
| | DYWGKGTQVTVSS | |
| 37 | MAQVQLVESGGGTVQAGGSLRLSCTASGRGRNAMGWY | I11 |
| | RQVPGGKRELVASINRGGTTDYTDSVKDRFTISRDSAKN | |
| | TVYLQMDSLQPEDTAVYYCNAESTVIPGHIYDYWGRGTQ | |
| | VTVSS | |
| 38 | GSGSGSGSGSGSGSGS | Binding peptide |
| 39 | LPETG | Sortase |
| | | recognition site |

As is well known to the person skilled in the art, the combination of CDR1, CDR2 and CDR3 is sufficient to define an antigen-binding site.

For the purposes of the invention, the term “CDR” (for “complementarity determining region”) refers to amino acid sequences which together define the binding affinity and specificity of the natural Fv region of a native immunoglobulin binding site.

According to a specific implementation of the invention, the PCPE-1-specific ligand is a nanobody whose polypeptide sequence includes three complementarity-determining regions (CDRs) with PCPE-1, characterized in that among these three CDRs, at least one CDR has at least 80% identity with one of the sequences SEQ ID NO. 1 to SEQ ID NO. 15.

According to another specific implementation, of these three CDRs designated CDR1, CDR2 and CDR3, each CDR is defined as follows:
i. CDR1 has at least 80% identity with one of the following sequences: SEQ ID NO. 1, 4, 7, 10 or 13;
ii. CDR2 has at least 80% identity with one of the following sequences: SEQ ID NO. 2, 5, 8, 11 or 14; and
iii. CDR3 has at least 80% identity with one of the following sequences: SEQ ID NO. 3, 6, 9, 12 or 15.

### According to Another Particular Implementation:

the CDR1 of the nanobody has at least 80%, 85%, 90%, 95%, 98%, 99% or 100% identity with at least one of the CDR1 sequences mentioned in (i) above, and
the CDR2 of the nanobody has at least 80%, 85%, 90%, 95%, 98%, 99% or 100% identity with at least one of the CDR2 sequences mentioned in (ii) above, and
the CDR3 of the nanobody has at least 80%, 85%, 90%, 95%, 98%, 99% or 100% identity with at least one of the CDR3 sequences mentioned in (iii) above.

The percentages of identity referred to in the present invention are determined after optimal alignment of the sequences to be compared, which may therefore comprise one or more additions, deletions, truncations and/or substitutions.

This percentage of identity can be calculated by any sequence analysis method well known to the person skilled in the art.

The percentage of identity can be determined after global alignment of the sequences to be compared, taken as a whole, over their entire length. As well as manually, the global sequence alignment can be determined using the algorithm of Needleman and Wunsch (1970).

For nucleotide sequences, sequence comparison can be carried out using any software program well known to the skilled person, such as Needle. The parameters used can in particular include: “Gap Open” equal to 10.0, “Gap Extend” equal to 0.5 and the EDNAFULL matrix (EMBOSS version of NCBI NUC4.4).

For amino acid sequences, sequence comparison can be carried out using any software program well known to the person skilled in the art, such as Needle. The parameters used can in particular include: “Gap Open” equal to 10.0, “Gap Extend” equal to 0.5 and the BLOSUM62 matrix. Preferably, the percentage of identity defined within the scope of the present invention is determined by means of a global alignment of the sequences to be compared over their entire length.

According to another implementation, the ligand specific for the PCPE-1 glycoprotein is a nanobody which comprises three complementarity determining regions (CDRs) with PCPE-1, said three CDRs exhibiting at least 80% sequence identity with the following sequence combinations:
a) CDR1: SEQ ID NO. 1, CDR2: SEQ ID NO. 2 and CDR3: SEQ ID NO. 3; or
b) CDR1: SEQ ID NO. 4, CDR2: SEQ ID NO. 5 and CDR3: SEQ ID NO. 6; or
c) CDR1: SEQ ID NO. 7, CDR2: SEQ ID NO. 8 and CDR3: SEQ ID NO. 9; or
d) CDR1: SEQ ID NO. 10, CDR2: SEQ ID NO. 11 and CDR3: SEQ ID NO. 12; or
e) CDR1: SEQ ID NO. 13, CDR2: SEQ ID NO. 14 and CDR3: SEQ ID NO. 15.

### According to a Particular Implementation:

the three CDRs of the nanobody show at least 80%, 85%, 90%, 95%, 98%, 99% or 100% identity with the CDR sequences mentioned in (a) above, or
the three nanobody CDRs have at least 80%, 85%, 90%, 95%, 98%, 99% or 100% identity with the CDR sequences mentioned in (b) above, or
the three CDRs of the nanobody have at least 80%, 85%, 90%, 95%, 98%, 99% or 100% identity with the CDR sequences mentioned in (c) above, or
the three CDRs of the nanobody have at least 80%, 85%, 90%, 95%, 98%, 99% or 100% identity with the CDR sequences mentioned in (d) above, or
the three CDRs of the nanobody have at least 80%, 85%, 90%, 95%, 98%, 99% or 100% identity with the CDR sequences mentioned in (e) above.

According to another implementation, the ligand specific for the PCPE-1 glycoprotein is a nanobody which comprises three complementarity determining regions (CDRs) with PCPE-1, said three CDRs having the following sequences:
a) CDR1: SEQ ID NO. 1, CDR2: SEQ ID NO. 2 and CDR3: SEQ ID NO. 3; or
b) CDR1: SEQ ID NO. 4, CDR2: SEQ ID NO. 5 and CDR3: SEQ ID NO. 6; or
c) CDR1: SEQ ID NO. 7, CDR2: SEQ ID NO. 8 and CDR3: SEQ ID NO. 9; or
d) CDR1: SEQ ID NO. 10, CDR2: SEQ ID NO. 11 and CDR3: SEQ ID NO. 12; or
e) CDR1: SEQ ID NO. 13, CDR2: SEQ ID NO. 14 and CDR3: SEQ ID NO. 15.

According to another implementation of the invention, the ligand specific for the PCPE-1 glycoprotein is a nanobody whose polypeptide sequence has at least 80% identity with one of the sequences SEQ ID NO. 16 to SEQ ID NO. 20.

In particular, this nanobody has a polypeptide sequence having at least 80%, 85%, 90%, 95%, 98%, 99% or 100% identity with the sequences SEQ ID NO. 16 to SEQ ID NO. 20.

Preferably, the substitutions observed in the polypeptide sequences will be placed in the domains outside the CDRS, referred to as the nanobody's “framework” regions.

More preferentially, the CDRs are conserved and show 100% identity with the sequences mentioned above in (a) for SEQ ID NO. 16; in (b) for SEQ ID NO. 17; in (c) for SEQ ID NO. 18; in (d) for SEQ ID NO. 19; and in (e) for SEQ ID NO. 20.

According to a preferred implementation, the nanobody of the invention has a polypeptide sequence consisting of the sequence SEQ ID NO. 16; or SEQ ID NO. 17; or SEQ ID NO. 18; or SEQ ID NO. 19; or SEQ ID NO. 20.

### Nanobody Combination

In another embodiment, the present invention discloses the association of these nanobodies within a single diabody or tribody structure. This association is preferably achieved by adding a binding agent between the protein sequences of each nanobody. The person skilled in the art will know how to select the most suitable binding agent, particularly from those disclosed in the review by Kwon, 2019. According to a preferred implementation, the GS₈ sequence (GSGSGSGSGSGSGSGS, SEQ ID NO. 38) is used as binding agent.

According to one embodiment of the invention, the specific ligand of the PCPE-1 glycoprotein consists of a combination of two or three nanobodies as described above, that is, a diabody or a tribody, optionally comprising one or two binding agents, in particular one or two binding peptides (one for a diabody, two for a tribody).

According to a particular implementation of the invention, a diabody consists of the combination of a nanobody with CUB1-binding activity and another with CUB2-binding activity, it being understood that each nanobody can be positioned either upstream or downstream of the binding peptide.

In particular, a diabody according to the invention may consist of any combination of two polypeptide sequences each having at least 80% or 90% sequence identity with one of the sequences SEQ ID NO. 16, 17, 18, 19 or 20, optionally further comprising a binding agent.

More specifically, a diabody will consist of any combination of two polypeptide sequences, each having a sequence chosen from sequences SEQ ID NO. 16 to SEQ ID NO. 20, optionally linked by a binding agent, in particular a binding peptide.

In particular, this combination of two nanobodies (diabodies) may have a polypeptide sequence at least 80% or 90% identical to sequence SEQ ID NO. 21, and in particular at least 95%, 98%, 99% or 100% identity with sequence SEQ ID NO. 21.

More preferentially, in this diabody, the CDRs of the nanobodies are conserved and show 100% identity with the sequences mentioned above in (a) for SEQ ID NO. 16 (H4) and in (c) for SEQ ID NO. 18 (15).

According to the present invention, this type of diabody may correspond to 15-linker-H4 or H4-linker-15. An example of the amino acid sequence for the H4-linker-15 diabody corresponds to sequence SEQ ID NO. 21.

According to a preferred implementation, a diabody according to the invention has a polypeptide sequence consisting of the sequence SEQ ID NO. 21.

A further object of the invention relates to a nucleic acid comprising a nucleic acid sequence encoding a nanobody or a combination of two or three nanobodies according to the present invention.

In a particular embodiment, the nucleic acid according to the invention comprises or consists of a nucleic acid sequence encoding a nanobody defined by one of the amino acid sequences SEQ ID NO. 16 to 20 or coding for a diabody defined by sequence SEQ ID NO. 21.

This nucleic acid is a DNA or RNA molecule, which can be included in any suitable vector, such as a plasmid, cosmid, episome, artificial chromosome, phage or viral vector.

The term “vector” refers to the vehicle whereby the DNA or RNA sequence can be introduced into a host cell, so as to transform the host and promote expression (e.g. transcription and translation) of the introduced nucleic acid sequence. Such a vector advantageously comprises regulatory elements, such as a promoter, activator, terminator, etc., to induce polypeptide expression. As a result, another object of the invention relates to a vector comprising a nucleic acid according to the invention.

Another object of the invention is a host cell that has integrated a vector as described above, and can thus express the nanobody, diabody or tribody according to the invention.

### Uses of PCPE-1-Specific Ligands According to the Invention

The present invention also relates to PCPE-1-specific ligands as defined above for use as contrast agents in non-invasive medical imaging, for use in diagnostic methods and for use as drugs.

Moreover, the ligands disclosed in the present invention, possessing both PCPE-1 binding and inhibitory activity, are ideal candidates for a so-called theranostic approach.

Finally, it relates to a pharmaceutical composition comprising one of these ligands in association with a pharmaceutically acceptable vehicle.

The compounds according to the invention can be used in immunoassays (ELISA, Western-blot, immunofluorescence, immunohistochemistry) to detect PCPE-1 in biological fluids or tissues, in vitro or ex vivo.

In particular, the present invention relates to a PCPE-1 glycoprotein antagonist ligand as described above, for use as a drug.

The present invention also relates to a PCPE-1 glycoprotein antagonist ligand as described above, for therapeutic use thereof in the treatment of cancer or fibrosis, particularly cardiac fibrosis.

Fibrosis, also known as sclerosis, occurs when tissue is substantially destroyed, or when inflammation occurs in an area where tissue cannot regenerate. Fibroses are pathologies characterized by excessive synthesis and deposition of extracellular matrix (ECM), leading to pathological scarring and rigidification that can lead to death when vital organs are affected (heart, liver, lung, etc.). There are several types of fibrosis: cardiac fibrosis, pulmonary fibrosis, hepatic fibrosis, renal fibrosis, muscular fibrosis, etc.

Cancer is also a disease in which collagen plays a significant role, although this role is complex and dependent on tumor type (Shi et al., 2021).

The present invention also relates to a method of treating cancer or fibrosis, in particular cardiac fibrosis, comprising administration to a patient suffering from cancer or fibrosis of a PCPE-1 antagonist ligand, inhibiting its activity, as described above.

In the context of the invention, a “patient” refers to a human or non-human mammal, such as a rodent (rat, mouse, rabbit), primate (chimpanzee), feline (cat), or canine (dog). Preferably, the patient is a human being, in particular suffering from cancer or fibrosis, and in particular cardiac fibrosis.

### Ligands Coupled to Detectable Markers

The present invention also relates to a ligand specific for the PCPE-1 glycoprotein as defined above, characterized in that it is coupled to a marker detectable in medical imaging.

“PCPE-1-specific ligand coupled to a detectable marker” means herein that the detectable marker is linked, directly or indirectly, to the ligand, or is incorporated into the ligand. In particular, the detectable marker can be linked to the ligand by substitution, complexion or chelation. Within the meaning of the present invention, a “detectable marker” refers to a compound that produces a detectable signal. When combined with a tracer, it can be used to monitor the fate of the tracer in the body. A detectable marker here refers in particular to a marker detectable in medical imaging.

The detectable marker used can be an MRI contrast agent, a scintigraphy contrast agent, an X-ray imaging contrast agent, an ultrasound contrast agent or an optical imaging contrast agent.

Examples of detectable markers include radioelements, fluorophores such as fluorescein, Alexa, cyanine; chemiluminescent compounds such as luminol; bioluminescent compounds such as luciferase or alkaline phosphatase; contrast agents such as nanoparticles or Gadolinium; and quantum dots.

The choice of the appropriate detectable marker, which depends on the detection system used, is a matter for the person skilled in the art.

The marking can be oriented, via the introduction of a terminal cysteine, or a recognition sequence by sortase or another ligase. The marking can also be achieved by direct coupling after lysine activation.

The detectable marker is in particular a fluorophore, a chromophore or luminescent compound or an antibody-detectable label. It can also be a chelating agent, such as NODAGA, which is then coupled to a radioisotope, such as Ga⁶⁸.

Advantageously, the ligand modified in this way retains its ability to bind to PCPE-1.

The PCPE-1-specific ligand coupled to a detectable marker can be used to detect PCPE-1 protein in an immunoassay or in cell culture, as shown in Example 5.

The present invention also relates to the use of a PCPE-1-specific ligand coupled to a detectable marker as defined above, for in vivo monitoring of a pathological condition by medical imaging.

In particular, the invention relates to the use of said PCPE-1-specific ligand coupled to a detectable marker as a contrast agent in medical imaging, in particular non-invasive, in vivo medical imaging.

For the purposes of this invention, a contrast agent is defined as a substance which, when administered to the body, enables organs or structures (tissue, cell, receptor) to be marked in a detectable way, which, without a contrast agent, would be difficult or impossible to see in medical imaging.

Advantageously, the PCPE-1-specific ligand coupled to the marker has pharmacokinetic characteristics which are compatible with its use as a contrast agent (rapid clearance but sufficient persistence to enable imaging, renal clearance, no accumulation in healthy animals).

This is shown in particular in Example 6. If required, these properties can be modulated by PEGylation, or other modification of the molecules' charge or lipophilicity.

The present invention also relates to a method for medical imaging, in particular non-invasive in vivo medical imaging, wherein a PCPE-1-specific ligand coupled to a detectable marker as defined above is administered to a patient, and the patient is then subjected to a medical imaging protocol.

It also relates to the use of a PCPE-1-specific ligand coupled to a detectable marker as defined above, for the manufacture of a contrast agent useful for medical imaging, in particular non-invasive in vivo medical imaging.

### Theranostic Use

Theranostics is a neologism derived from the terms therapy and diagnosis, and corresponds to a medical approach that emphasizes the simultaneous development of diagnostic and therapeutic aspects. In particular, the aim is to use compounds that can be used both to visualize a patient's clinical situation in vivo, and to treat the condition in question with the same compound. The PCPE-1-specific ligands according to the invention, in particular those inhibiting PCPE-1 activity and coupled to a detectable marker, are highly suitable for theranostic use. Thus, the present invention relates to a PCPE-1-specific ligand coupled to a detectable marker, for theranostic use thereof in the treatment and in vivo monitoring of a pathological condition by medical imaging. Preferably, this ligand is a PCPE-1 antagonist.

### Detection Kit

The present invention also relates to a kit for determining the activity and/or amount of the PCPE-1 glycoprotein in a biological sample in vitro or ex vivo, comprising:
a ligand specific for the PCPE-1 glycoprotein according to the invention, and
detection reagents.

In particular, this PCPE-1-specific ligand will be coupled to a detectable marker.

A biological sample refers to any type of sample from a living body, in particular a patient's body, and in particular includes blood, serum, plasma, urine, cerebrospinal fluid and tears.

### EXAMPLES

### Materials and Methods

### Protein Production and Purification

The proteins PCPE-1 (human native or with 8his tag in C-terminal), Mini-procollagen I (Mini I, Twin-strep tag in N-terminal of α2 chain), Mini-procollagen II (Mini II, N-terminal 6His tag), Mini-procollagen III (Mini III, N-terminal C-myc tag), and BMP-1 (C-terminal flag tag) were produced in HEK 293-EBNA or 293-F cells and purified as previously disclosed. The CUB domains of PCPE-1 were prepared by limited proteolysis of CUB1NTR and CUB2NTR as disclosed by Kronenberg et al. CPIII-Long and PCPE-2 (with a 6-his N-terminal tag) were produced by transient transfection of HEK 293T cells and purified as previously disclosed.

The antigen used for nanobody selection is the CUB1CUB2 region of the PCPE-1 protein (corresponding to amino acids 1 to 279). It was cloned into the pHLsec vector, fused with a 6-His tag and an N-terminal HRV-3C protease cleavage sequence. It was then produced by transient transfection of 293-F cells, grown at 37° C., 125 rpm, 8% CO₂ in FreeStyle™ 293 medium (Gibco), following the procedure described by Pulido et al. CUB1CUB2 was purified on a Ni-excel column (5 mL; Cytiva), then treated with HRV-3C protease at 4° C. overnight to remove the histidine tag. After gel filtration with a HiLoad Superdex 75 16/600 SEC (size exclusion chromatography, Cytiva) column equilibrated with 20 mM HEPES pH 7.4, 0.3 M NaCl, the protein is stored at −80° C. in the same buffer. Protein concentration was determined using a Nanodrop 2000 (Thermo Scientific).

A biotinylated form of CUB1CUB2 was prepared for bio-panning and phage-ELISA. To achieve this, the sequence coding for the CUB1CUB2 region was cloned into the pHL-Avitag3 vector between the EcoRI and KpnI sites, then the protein was expressed in 293-T as above. After purification on Cobalt resin, 60 μM of CUB1CUB2avi was biotinylated with 1 μM GST-BirA (Biotin-protein ligase) in 50 mM bicine buffer, 300 mM potassium glutamate pH 8.3 in the presence of 10 mM ATP and 50 μM d-biotin for 5 h at 30° C. GST-BirA (cloned into the pGex vector, donated by Y. Zhao, STRUBI, Oxford, GB) was produced in E. Coli BL21 (DE3) pLysS bacteria. Purification on cobalt resin followed by gel filtration on a Superdex S75 column (20 mM HEPES buffer pH 7.4, 0.3 M NaCl) removed excess BirA and biotin, yielding a 95% pure, biotinylated protein.

### Nanobody Selection in Llamas

Llama nanobodies were generated on the platform of the Laboratoire Architecture et Fonction des Macromolécules Biologiques [Biological Macromolecular Architecture and Function Laboratory] (Marseille, France). Briefly, a Llama glama was immunized by five successive injections of 1 mg CUB1CUB2 at one-week intervals. After 39 days, the blood was collected, and the nanobody library was generated as previously described. Two bio-panning steps on 50 nM biotinylated CUB1CUB2 were used to enrich phages expressing nanobodies specifically recognizing CUB1CUB2. 48 clones (phagemid) were selected and their affinity for human PCPE-1 determined by phage ELISA. Positive clones were sequenced and their sequences aligned and analyzed using ESPript 3.0. Eight nanobodies were selected for further characterization: I1, I2, I3, I4, I5, I7, I10 and I11.

### Generation of Synthetic Nanobodies

The synthetic nanobodies were selected by Hybrigenics Services SAS. After three steps of biopanning on biotinylated CUB1CUB2 using the hsd2ab nanobody library (Moutel et al., 2016), 90 clones were tested by phage ELISA for their ability to bind to PCPE1. After sequencing, a library of 24 unique positive clones was obtained. Ten of them were selected for more detailed characterization: H1 to H10.

### Nanobody Production and Purification

Synthetic nanobodies were cloned into the pET29b (+) vector, fused with the pelB signal sequence and a C-terminal 6-His tag. The llama nanobodies were cloned into the pHEN6 vector following the pelB sequence and fused with a C-terminal 6-His tag.

To construct the diabody, the VHH-I5 nanobody was amplified by PCR with the addition of a NotI restriction site and a GS₈ sequence (GSGSGSGSGSGSGSGS, SEQ ID NO. 38) in the N-terminus.

The GS₈-VHH-I5 construct was then cloned following VHH-H4 into pET29b (+) by NotI/XhoI digestion to lead to the bivalent nanobody VHH-H4-GS₈-VHH-I5 (D1).

The resulting plasmids are then transformed into E. coli T7 express (NEB) bacteria. When bacterial density reaches OD600=0.8, protein expression is triggered by treatment with 0.1 mM IPTG (β-D-1-thiogalactopyranoside) and cells are incubated overnight at 28° C., in Terrific Broth medium containing 0.1% glucose. Cells are then harvested by centrifugation at 4000 rpm for 15 min at 4° C. and resuspended in TES buffer (200 mM Tris HCl pH 8.0, 500 mM Sucrose, 50 nM EDTA). The periplasmic fraction is prepared by osmotic shock and nanobodies are purified by affinity chromatography on nickel resin (Qiagen) and gel filtration (Superdex 75 16/600; Cytiva) in 20 mM HEPES, 0.3 M NaCl pH 7.4 buffer.

Some nanobodies were also cloned into the pHEN vector containing the LPETG sequence (recognition site for Sortase, SEQ ID NO. 39) and a 6-His label in the C-terminal position (donated by Dr. Leo Hanke, Karolinska Institut, Stockholm, Sweden), and produced and purified as disclosed in (Hanke et al., 2020). In parallel, 5 M Sortase A cloned into the pET30b vector (Addgene #51140), with a C-terminal 6-His tag, was produced and purified according to the supplier's instructions. The nanobodies were biotinylated at their C-terminal end using 50 μM Sortase A 5 M and 200 μM GGGK-biotin (Covalab) in 50 mM HEPES pH 7.5, 150 mM NaCl, 10 mM CaCl₂) buffer, for 2 h at 25° C. The 5 M Sortase A and excess nanobodies were removed on Ni-NTA resin (Qiagen; 2 mL), followed by excess biotin on Zeba spin (7K MWCO, Thermo Fisher Scientific).

### Analyses by SPR

Surface plasmon resonance (SPR) experiments were carried out on a Biacore T200 (Cytiva). The ligand proteins, in solution in 10 mM HEPES pH 7.4 buffer for PCPE-1 or 10 mM sodium acetate pH 4.5 buffer for mini-procollagen III and streptavidin, are immobilized on CM5 chips by amine coupling. Analytes are diluted in running buffer (10 mM HEPES pH 7.4, 0.15 M NaCl, 5 mM CaCl₂), 0.05% P20) and injected at a flow rate of 30 or 50 μL/min. Competition, epitope binning and kinetics experiments were all carried out at 25° C. Regeneration was achieved by successive injection of 2 M guanidine chloride and 0.5 M EDTA for 30 sec. Sensorgrams were analyzed with Biacore T200 Evaluation v3.2.1 (Cytiva), using the most suitable models. IC50 was determined by non-linear regression using GraphPad Prism (v8.2.1).

### Activity Tests

To characterize the effects of nanobodies on PCPE-1 activity, 400 nM CPIII-Long (or Mini I or Mini II) were incubated for 1 h at 37° C. with 2.5 nM BMP-1 and 75 nM PCPE-1 (when present) in the presence or absence of 2 μM nanobodies in the following buffer: 20 mM HEPES pH 7.4, 0.15 M NaCl, 5 mM CaCl2, 0.02% n-octyl-β-D-glucopyranoside. Samples were analyzed by SDS-PAGE on 4-20% gradient gels (Criterion; Biorad) and Instant Blue staining (Euromedex). The level of activation by PCPE-1 was assessed as the ratio between the intensity of the C-propeptide band and the intensity of all long CPIII bands (cleaved and uncleaved), after normalization with the BMP-1 condition alone, using ImageQuantTL software (Cytiva).

### Effect of Nanobodies on C-Terminal Maturation of Fibrillar Collagens In Vitro

Rat heart fibroblasts were cultured in DMEM medium, 10% SVF (Eurobio), 1% AAS (Antibiotic-antimycotic solution; Thermo Fisher) at 37° C., 5% CO₂. At 70% cell confluence, serum was removed and replaced with serum-free medium containing PCPE-1 (5 μg-mL⁻¹) or/and 15 μg·mL⁻¹ nanobodies. After 48 h of culture, the supernatants are harvested and the procollagen is analyzed by western-blotting using an anti-C-propeptide antibody (LF41, donated by Dr. Larry W. Fisher, Bethesda, USA). Bands are quantified using ImageQuantTL software (Cytiva).

Human skin fibroblasts (donated by the tissue and cell bank of the Edouard Herriot Hospital, Lyon) were cultured in the same way.

### Analyses by ELISA

100 ng of anti-PCPE-1 antibody in solution in carbonate buffer pH 9.6 is coated onto the bottom of a microplate overnight at 4° C. After blocking with a 1% BSA solution, increasing amounts of PCPE-1 (purified human protein) are added and left in contact for 1.5 hours at room temperature. PCPE-1 is detected by addition of biotinylated VHH, e.g. VHH-I5 (100 ng) for 1 h at room temperature, followed by addition of extravidin-HRP and detection using TMB solution. To detect PCPE-1 in blood, human plasma samples are diluted to 50:1 and analyzed as above.

### Marking of VHH-H4 and Study of its Characteristics

VHH-H4 is bioconjugated with NODAGA and then marked with gallium 68 using the procedure disclosed in (Renard et al, 2020). Its biodistribution is assessed in rats after IV injection of 25 μg (12 MBq). To this end, 9 blood samples (150-200 μL) were taken during the 2.5 hours following injection, after which the animal was euthanized. Tissue samples were analyzed by gamma counting.

### Statistical Analysis

Unless otherwise stated, data represent the mean±standard deviation of at least three independent experiments, calculated using Graphpad Prism 8.2 software

### Results

### Example 1. Selection and Characterization of Nanobodies Directed Against PCPE-1

FIG. 1 shows the results of the selection of PCPE-1-binding nanobodies from the llama nanobody library or a synthetic library.

As the CUB1 and CUB2 domains of PCPE-1 are necessary and sufficient for its activity, we chose to use as antigen a protein containing these two domains, but lacking the NTR domain. The labels used for purification were eliminated to avoid the selection of non-specific ligands. Two strategies were pursued in parallel: in vivo selection of llama nanobodies by animal immunization, and in vitro selection from a library of synthetic nanobodies.

For the immune library, nanobodies were generated by five successive injections of recombinant CUB1CUB2 into a llama, followed by two biopanning steps. 48 clones were analyzed by phage-ELISA, resulting in 11 unique clones after sequencing. These 11 clones can be classified into six families according to their CDR3. The first family is the most represented, with 5 clones, while the others have only one or two variants. 3 members of family 1 (VHH-I1, VHH-I5 and VHH-I7) and one member of each of the other families were selected, produced in E. coli periplasm and purified. Their affinity for PCPE-1 was measured by surface plasmon resonance (SPR) (FIG. 1A). All except VHH-I10 bind to PCPE-1 (Table 2). Nanobodies belonging to the same family behave very differently. In family 1, the interaction of VHH-I1 with PCPE-1 appears less stable than those of VHH-I5 and -I7, leading to a significantly higher dissociation constant (Table 2). Among other llama nanobodies, only VHH-I3 is a potent ligand for PCPE-1, with dissociation constants in the nanomolar range. Synthetic nanobodies were selected using three cycles of phage display, then 90 clones were randomly selected and tested by phage ELISA for their ability to bind to PCPE-1. 32% of clones gave a positive signal, corresponding to 24 unique clones after sequencing. 7 of these 24 clones also gave a positive signal when tested against immobilized CUB1NTR. 10 clones (including these 7) were finally produced and purified, then their affinity for PCPE-1 was measured by SPR (FIG. 1B). Of the 10, only half show a clear interaction with PCPE-1 when injected at 200 nM, with the best results obtained with VHH-H4 and VHH-H10 (FIG. 1B), whose K_{D}s are in the nanomolar range (Table 2).

### Measurement of the K_{D} Affinity Constant of the Various Nanobodies Studied

Nanobodies from the synthetic nanobody library are designated by names beginning with the letter H; nanobodies from the serum of an immunized llama are designated by names beginning with I.

FIG. 1A shows the PCPE-1 binding activity of eight nanobodies derived from llama serum: I1, I2, I3, I4, I5, I7, I10 and I11.

FIG. 1B shows the PCPE-1 binding activity of the following ten synthetic nanobodies: H1 to H10.

Table 2 below shows the K_{D} affinity constants of seven llama-derived nanobodies (as mentioned above, the I10 nanobody does not bind at all), and of the ten synthetic nanobodies.

TABLE 2

| | K_{D} (nM) |
|---|---|
| I1 | >250 |
| I2 | 154 |
| I3 | 16.0 |
| I4 | >250 |
| I5 | 7.2 |
| I7 | 12.8 |
| I11 | 161 |
| H1 | >250 |
| H2 | >250 |
| H3 | >250 |
| H4 | 30 |
| H5 | >250 |
| H6 | >250 |
| H7 | 152 |
| H8 | >250 |
| H9 | >250 |
| H10 | 18 |

Affinity constants below 100 nM are representative of strong binding; this is the case for the following nanobodies: 13, 15, 17, H4 and H10.

### Example 2. Determination of Regions Recognized by Nanobodies

FIG. 2 shows the results of the determination of PCPE-1 regions recognized by antibodies. Nanobody co-injection competition experiments with the CUB1 or CUB2 domain of PCPE-1 were carried out (FIG. 2A). When mixed with CUB2, nanobodies I3, I5 and I7 proved unable to bind to PCPE-1, while co-injection with CUB1 had virtually no effect, indicating that these nanobodies interact primarily with the CUB2 domain of PCPE-1. In contrast, the interaction of H4 and H10 synthetic nanobodies with PCPE-1 is prevented by co-injection with CUB1 and not with CUB2, indicating that they interact with the CUB1 domain.

For the best ligands in each library (I5 and H4), we then determined whether the other nanobodies possessed the same epitope or were able to bind simultaneously to PCPE-1. To achieve this, the PCPE-1 chip was first saturated with nanobody-I5 or -H4. While no change in signal is observed when VHH-I5 is subsequently co-injected with the other llama nanobodies, co-injection of VHH-I5 and VHH-H4 or -H10 leads to a sharp increase in signal (FIG. 2B), indicating that they bind simultaneously to distinct PCPE-1 sites. Similarly, when the surface is first saturated with VHH-H4 (FIG. 2C), the llama nanobodies are still able to interact with PCPE-1, while VHH-H10 can no longer bind. These results confirm that the two libraries have different epitopes, but that within a library, epitopes are rather conserved.

PCPE-2 is a protein in the same family as PCPE-1, with a 43% identical amino acid sequence. It shares certain activities with PCPE-1, but also has very different specific functions. The specificity of nanobody interaction for PCPE-1 versus PCPE-2 was analyzed by SPR. To achieve this, VHH-I5 and VHH-H4 were biotinylated at their C-terminus using sortase, then immobilized on a streptavidin surface. In both cases, interaction with PCPE-2 is virtually undetectable (K_{D}>1 μM), while PCPE-1 binds very strongly (K_{D}=1.5 nM (VHH-I5) and K_{D}=3.9 nM (VHH-H4), FIG. 2D-E and Table 3 below). The affinity of VHH-I5 and -H4 for PCPE-1 is therefore at least 250 times greater than for PCPE-2.

TABLE 3

| | | kₐ | k_{d} | K_{D} | Rₘₐₓ | |
|---|---|---|---|---|---|---|
| 1:1 binding model | | (M⁻¹ s⁻¹) | (s⁻¹) | (nM) | (RU) | X² |
| I5 | PCPE-1 | 9.37⁵ 10⁵ | 1.36 10⁻³ | 1.45 | 2643 | 206 |
| | PCPE-2 | — | — | >1000 | — | — |
| H4 | PCPE-1 | 5.04 10⁶ | 1.95 10⁻² | 3.88 | 1093 | 73.8 |
| | PCPE-2 | — | — | >1000 | — | — |

### Example 3: Antagonistic Inhibitory Effects of Nanobodies According to the Invention on PCPE-1

FIG. 3 shows the results of the inhibitory effect of the nanobodies according to the invention on PCPE-1 activity. PCPE-1 is active via its direct interaction with procollagen C-propeptides. We therefore observed whether nanobodies were able to prevent PCPE-1 binding to a mini-procollagen III (Mini III, composed of the C-terminal domains of procollagen III: end of triple helix, C-telopeptide and C-propeptide) using SPR competition experiments.

The results show that VHH-I3, -I5, -I7, -H4 and -H10 inhibit PCPE-1 binding to Mini III, while VHH-I1, -I2, -I4 and -I11 have no effect (FIG. 3A).

VHH-H4, -H10 and VHH-I5 are the most effective, with 50-75% inhibition at 250 nM. What is more, their antagonistic effects are additive, and co-injection of a mixture of VHH-I5 and VHH-H4 with PCPE-1 virtually eliminates its interaction with Mini III. This inhibitory effect increases with concentration, with IC50s estimated at around 45 nM (VHH-H4), 24 nM (VHH-I5) and 20 nM (mix of VHH-I5 and VHH-H4) in the presence of 5 nM PCPE-1. However, while complete inhibition is achieved at high concentrations of VHH-H4, the addition of VHH-I5 alone does not fully block the PCPE-1/Mini III interaction, whereas inhibition is complete when VHH-H4 and VHH-I5 are combined.

Since the nanobodies prevent PCPE-1 binding to procollagens, the next step was to see whether they also inhibit PCPE-1 activity in vitro. CPIII-Long (a model substrate for procollagen III) is cleaved by incubation with the BMP-1 protease, and this cleavage is increased in the presence of PCPE-1 (FIG. 3B, 2.4-fold increase under these conditions). The addition of VHH-I1, VHH-I2, VHH-I4 or VHH-I11 has very little effect on PCPE-1 activity. Conversely, the addition of VHH-I3, VHH-I5, VHH-I7, VHH-H4 or VHH-H10 leads to a 25-45% drop in activation, which is totally inhibited by the simultaneous use of VHH-I5 and VHH-H4. It should also be noted that the basal activity of BMP-1 does not appear to be altered by the presence of VHHs, despite the fact that BMP-1 also contains three CUB domains.

Finally, nanobodies were evaluated in a cell-based test for their ability to modulate procollagen I cleavage.

To mimic fibrotic conditions, rat heart fibroblasts were treated with recombinant human PCPE-1 protein (FIG. 3C, D). The addition of PCPE-1 to the medium increases the release of procollagen I C-propeptide by a factor of around 2.5 on average. The presence of VHH-I5 and VHH-H4 inhibits the effect of PCPE-1 (by around 40%), as shown by the decrease in C-propeptide (FIG. 3C). Clearly, joint treatment with VHH-I5 and VHH-H4 completely blocks the effect of exogenous PCPE-1, with a return to basal levels of C-propeptide release. Moreover, treatment of cells with VHHs alone (in the absence of added PCPE-1) has no effect on C-propeptide release.

All these results suggest that the selected ligands are potent PCPE-1 antagonists, capable of blocking PCPE-1-mediated stimulation of procollagen C-terminal maturation.

### Example 4: Construction and Characterization of a Diabody (Divalent Nanobody)

The previous results suggest that combining 15 and H4 nanobodies in the same polypeptide chain could further enhance their affinity for PCPE-1 through site cooperativity.

FIG. 4 shows the results obtained for the diab-D1 diabody of SEQ ID NO. 21, produced in bacteria. The interaction of diab-D1 with PCPE-1 was assessed by SPR, by injecting increasing concentrations of diab-D1 onto immobilized PCPE-1. The benefit of VHH fusion is clearly visible in the avidity it provides, manifested by an improved dissociation constant (K_{D}=0.32 nM) and much slower dissociation (FIG. 4A).

Moreover, competition experiments were carried out to determine whether diab-D1 had a more powerful antagonistic effect than the nanobodies used individually (FIG. 4B). The IC50 calculated for diab-D1 is estimated at 3.5 nM, substantially lower than that for VHH-I5 and VHH-H4 alone or co-injected (5 to 13-fold) under the same conditions. Similarly, the addition of diab-D1 completely blocks the stimulation of CPIII-Long cleavage by PCPE-1 (FIG. 4C).

### Effect of Diab-D1 on Procollagen I and II Cleavage

The diab-D1 antibody is also able to block PCPE-1 activity on other fibrillar collagens. Indeed, the addition of diab-D1 inhibits the activation of Miniprocollagen I (Mini I) and II (Mini II) cleavage by PCPE-1, as shown in FIG. 4D.

### Modulation of Collagen Maturation by Diab-D1

In cell culture (FIG. 4E, 4F), diab-D1 is able to completely block the effect of PCPE-1 addition on the cleavage of endogenous procollagen I by rat heart fibroblasts. Moreover, and in contrast to individual VHHs, the diabody also inhibits C-propeptide cleavage in the absence of added PCPE-1.

The antibody also inhibits the cleavage of procollagen I produced by human skin fibroblasts, whether in the basal state or under “fibrotic” conditions mimicked by TGF-beta stimulation, or in the presence of added exogenous PCPE-1.

This is shown in FIGS. 4G and 4H, which show immunodetection of procollagen I and its C-terminal cleavage products in the culture medium of human skin fibroblasts, after 72 h of culture in the absence or presence of PCPE-1, TGF-beta and diab-D1.

Other measurements were carried out by immunofluorescence on cultured human fibroblasts (results not shown). The matrix deposited by the cells was also found to be affected, since the addition of diab-D1 led to a decrease in collagen deposition detected by immunofluorescence.

### Example 5: Nanobody Coupled to a Detectable Marker

Nanobodies containing the LPETG sequence (SEQ ID NO. 39) were coupled to biotin using Sortase as disclosed above. Nanobodies coupled in this way can be bound to streptavidin, enabling detection of PCPE1 in a complex mixture by SPR, ELISA or Western blot.

FIG. 5A shows the SPR study of the interaction of PCPE-1 with the biotinylated H4 nanobody, captured on a streptavidin-coupled chip.

FIG. 5B shows PCPE-1 detection using a sandwich ELISA. The sample containing PCPE-1 is captured by an anti-PCPE-1 antibody. The addition of biotinylated VHH-I5 enables its detection, using streptavidin coupled to HRP or a fluorophore such as Alexafluor488. The method is highly sensitive and easily detects 0-1 ng PCPE-1. This makes it possible to determine the amount of PCPE-1 present in human plasma, which is around 510±70 ng/ml.

Alternatively, PCPE-1 can be detected by sandwich ELISA between H4 and I5.

The nanobodies were also coupled to a radioactive marker, NODAGA-Ga⁶⁸.

To achieve this, VHH-H4 was first bioconjugated with NODAGA, then marked with gallium 68 according to the procedure disclosed in (Renard, 2020).

The biodistribution and pharmacokinetics of the nanobody were then evaluated in rats after intravenous injection of 25 μg (12 MBq) of H4-Ga⁶⁸, to determine the molecule's elimination pathways and areas of accumulation.

The results are shown in FIG. 6A (pharmacokinetics) and 6B (biodistribution in the main organs: heart, lungs, blood, liver, spleen, kidneys and bladder).

The H4-Ga⁶⁸ nanobody can also be used to detect PCPE-1 on tissue sections and detected by phosphorimager: see FIG. 6C.

### BIBLIOGRAPHY

US20120270246
WO2017065206
WO2019080284
Henderson N C, Rieder F, Wynn T A. Fibrosis: from mechanisms to medicines. Nature. 2020 November; 587(7835):555-566. doi: 10.1038/s41586-020-2938-9.
Shi R, Zhang Z, Zhu A, Xiong X, Zhang J, Xu J, Sy M S, Li C. Targeting type I collagen for cancer treatment. Int J Cancer. 2022 Feb. 28. doi: 10.1002/ijc.33985
Kronenberg, D., Vadon-Le Goff, S., Bourhis, J. M., Font, B., Eichenberger, D., Hulmes, D. J. & Moali, C. (2009). Strong cooperativity and loose geometry between CUB domains are the basis for procollagen C-proteinase enhancer activity. J Biol Chem. 284, 33437-33446
Vadon-Le Goff, S., Kronenberg, D., Bourhis, J. M., Bijakowski, C., Raynal, N., Ruggiero, F., Farndale, R. W., Stocker, W., Hulmes, D. J. & Moali, C. Procollagen C-proteinase enhancer stimulates procollagen processing by binding to the C-propeptide region only. J. Biol. Chem. 286, 38932-38938 (2011).
Lagoutte P, Bettler E, Vadon-Le Goff S, Moali C. Procollagen C-proteinase enhancer-1 (PCPE-1), a potential biomarker and therapeutic target for fibrosis. Matrix Biol Plus. 2021 Apr. 20; 11:100062. doi: 10.1016/j.mbplus.2021.100062.
Lagoutte, P., Oudot, A., Dussoyer, M., Goncalves, V., Bouchot, O., Vandroux, D., Bellaye, P. S., Moali, C. & Vadon-Le Goff, S. Procollagen C-Proteinase Enhancer 1 (PCPE-1) is a marker of myocardial fibrosis and impaired cardiac function in a murine model of pressure overload. bioRxiv 2021; doi: https://doi.org/10.1101/2021.03.05.434071
Désogère P, Montesi S B, Caravan P. Molecular Probes for Imaging Fibrosis and Fibrogenesis. Chemistry. 2019 Jan. 24; 25(5):1128-1141. doi: 10.1002/chem.201801578.
Muyldermans S. A guide to: generation and design of nanobodies. FEBS J. 2021 April; 288 (7): 2084-2102. doi: 10.1111/febs.15515.
Gebauer M, Skerra A. Engineering of binding functions into proteins. Curr Opin Biotechnol. 2019 December; 60:230-241. doi: 10.1016/j.copbio.2019.05.007. Epub 2019 Jun. 14. PMID: 31207556.
Needleman S B, Wunsch C D. A general method applicable to the search for similarities in the amino acid sequence of two proteins. J Mol Biol. 1970 March; 48(3):443-53. doi: 10.1016/0022-2836(70)90057-4. PMID: 5420325.
Kwon N Y, Kim Y, Lee J O. Structural diversity and flexibility of diabodies. Methods. 2019 Feb. 1; 154:136-142. doi: 10.1016/j.ymeth.2018.09.005. Epub 2018 Sep. 25. PMID: 30261312.
Pulido D, Sharma U, Vadon-Le Goff S, Hussain S A, Cordes S, Mariano N, Bettler E, Moali C, Aghajari N, Hohenester E, Hulmes D J S. Structural Basis for the Acceleration of Procollagen Processing by Procollagen C-Proteinase Enhancer-1. Structure. 2018 Oct. 2; 26(10):1384-1392.e3. doi: 10.1016/j.str.2018.06.011
Moutel S, Bery N, Bernard V, Keller L, Lemesre E, de Marco A, Ligat L, Rain J C, Favre G, Olichon A, Perez F. NaLi-H1: A universal synthetic library of humanized nanobodies providing highly functional antibodies and intrabodies. Elife. 2016 Jul. 19; 5: e16228. doi: 10.7554/eLife.16228.
Hanke L, Vidakovics Perez L, Sheward D J, Das H, Schulte T, Moliner-Morro A, Corcoran M, Achour A, Karlsson Hedestam G B, Hällberg B M, Murrell B, McInerney G M. An alpaca nanobody neutralizes SARS-CoV-2 by blocking receptor interaction. Nat Commun. 2020 Sep. 4; 11(1):4420. doi: 10.1038/s41467-020-18174-5.
Renard E, Dancer P A, Portal C, Denat F, Prignon A, Goncalves V. Design of Bimodal Ligands of Neurotensin Receptor 1 for Positron Emission Tomography Imaging and Fluorescence-Guided Surgery of Pancreatic Cancer. J Med Chem. 2020 Mar. 12; 63(5):2426-2433. doi: 10.1021/acs.jmedchem.9b01407. Epub 2020 Jan. 2. PMID: 31855417.

1. A ligand specific for the PCPE-1 glycoprotein, characterized in that it is a nanobody.

2. The ligand specific for the PCPE-1 glycoprotein, characterized in that it inhibits its interaction with the C-terminal domain of procollagens.

3. The ligand specific for the PCPE-1 glycoprotein according to claim 2, characterized in that it is a nanobody.

4. The ligand specific for the PCPE-1 glycoprotein according to claim 1, characterized in that said nanobody comprises three complementarity determining regions (CDRs) with PCPE-1, and that among these three CDRs, at least one CDR has at least 80% identity with one of the sequences SEQ ID NO. 1 to SEQ ID NO. 15.

5. The ligand specific for the PCPE-1 glycoprotein according to claim 4, characterized in that each of the three CDRs designated CDR1, CDR2 and CDR3 is defined as follows:
i. CDR1 has at least 80% identity with one of the following sequences: SEQ ID NO. 1, 4, 7, 10 or 13;
ii. CDR2 has at least 80% identity with one of the following sequences: SEQ ID NO. 2, 5, 8, 11 or 14; and
iii. CDR3 has at least 80% identity with one of the following sequences: SEQ ID NO. 3, 6, 9, 12 or 15.

6. The ligand specific for the PCPE-1 glycoprotein according to claim 1, whose peptide sequence has at least 80% identity with one of sequences SEQ ID NO. 16 to SEQ ID NO. 20.

7. The ligand specific for the PCPE-1 glycoprotein consisting of a combination of two or three nanobodies according to claim 1, in particular a combination of two polypeptide sequences each having at least 80% identity with one of sequences SEQ ID NO. 16 to SEQ ID NO. 20, optionally further comprising one or two binding agents.

8. The ligand specific for the PCPE-1 glycoprotein according to claim 7, whose polypeptide sequence has at least 80% identity with sequence SEQ ID NO. 21.

9. A nucleic acid encoding a nanobody according to claim 1.

10. The ligand specific for the PCPE-1 glycoprotein according to claim 2, for use as a drug.

11. The ligand specific for the PCPE-1 glycoprotein according to claim 2, for its medical use in the treatment of cancer or fibrosis, in particular cardiac fibrosis.

12. The ligand specific for the PCPE-1 glycoprotein according to claim 1, characterized in that it is coupled to a detectable marker.

13. A use of a ligand specific for the PCPE-1 glycoprotein according to claim 12, for in vivo monitoring of a pathological condition by medical imaging.

14. The ligand specific for the PCPE-1 glycoprotein according to claim 12, for its theranostic use in the treatment and in vivo monitoring of a pathological condition by medical imaging.

15. A kit for determining the activity and/or amount of the PCPE-1 glycoprotein in a biological sample in vitro or ex vivo, comprising:
a ligand specific for the PCPE-1 glycoprotein according to claim 1, and
detection reagents.

16. A nucleic acid encoding a combination of two or three nanobodies according to claim 1.
